(19) United States
(12) Patent Application Publication
(10) Pub. No.: US 2006/0241566 A1
(43) Pub. Date: Oct. 26, 2006

(54) Nucleus Extraction from Spine Intervertebral Disc

(75) Inventors: Jon K. Moon, Edina, MN (US); Thomas J. McPeak, Shakopee, MN (US); Peter M DeLange, Waconia, MN (US); Christopher Szczech, Clearwater, MN (US); Robert A. Connor, Plymouth, MN (US); David Edward Pries, Saint Paul, MN (US); Sun-Young Choh, Minneapolis, MN (US); Megan M. Kruse, Arden Hills, MN (US)

Correspondence Address: ORTHOX, LLC, 207 N. CHESTNUT ST., #225, CHASKA, MN, 55318, US

(73) Assignee: Orthox, LLC, Chaska, MN (US)

(21) Appl. No.: 11/279,397

(22) Filed: Apr. 11, 2006

Related U.S. Application Data
(60) Provisional application No. 60/670,305, filed on Apr. 11, 2005.

(51) Int. Cl. A61M 1/00

(52) U.S. Cl. 604/540

(57) ABSTRACT

This invention proposes devices and methods directed to providing rapid and complete surgical removal of the nucleus from the spine intervertebral space. In addition, the invention protects the endplate tissue of vertebrae containing the disc and limits damage to the integrity of the disc annulus.

### FIELD OF THE INVENTION

This invention relates to devices and methods for use in interventions to restore spinal function. More specifically, the invention removes nucleus pulposus from the intact spine intervertebral disc during surgical therapy to treat herniation or degenerated discs.

### BACKGROUND OF THE INVENTION

Back and spinal ailments trouble thousands of Americans every year. In 2003 approximately 11 million people had impaired movement because of back pain, resulting in $80 billion of lost work and productivity. Back pain is a top cause of health care expenditures, amounting to $50 billion in the USA alone. However, only 2 percent of patients seek current implant therapies that create spinal fusion, and they typically do so only at an advanced stage of disease.

Disc degeneration is part of the natural process of aging and has been documented in approximately 30% of 30 year olds. As the population ages, it is even more common for individuals to have signs of disc degeneration. Disc degeneration is an expected finding over the age of 60.

Many back problems result from failure of the annulus (also called the disc annulus or outer fibrous ring) and from herniation of the nucleus pulposus (also called the disc nucleus) through the annulus of the intervertebral disc to compress the spinal cord or nerve roots. Currently, there are a limited number of treatments for these ailments. First, if the nucleus is still relatively intact, a physician can remove the herniating portion and leave the remaining nucleus in an effort to maintain the integrity and mobility of that spinal region. Successful surgery depends on integrity of the annulus and involves the assessed risk of additional future herniation. Or, physicians can remove much of the intervertebral disc with the intention of preventing future herniations by facilitating a fusion of adjacent discs.

These interventions are great advancements over treatments that were available just decades ago. But, they introduce several concerns and difficulties. One of the most difficult decisions that physicians face is to determine the amount of nucleus to remove. If too much is removed then mobility can be reduced, too little and the herniation may recur. There is also substantial risk of damage to the annulus that could impair healing. Procedures that remove the complete intervertebral disc, discectomy, damage the vertebral end plate. Due to the similar texture of the ligamentum flavum and the dura there is also concern of cutting into the dura, which could result in neurological complications. Finally, these procedures produce large amounts of scarring, which limits the scope of revision surgeries.

A new treatment uses intervertebral implants to replace the nucleus with materials that restore mobility and avoid adjacent segment deterioration without the risk of herniation. Manufacturers have developed implants to the point that several forms of the prostheses are in clinical trials. Although there are associated problems and difficulties, these implants are poised to be a major breakthrough treatment of failed intervertebral discs, particularly in young people. The implants are placed within the space defined by the annulus after as much of the nucleus as possible has been removed. Because the goal of the surgery is to restore mobility, the annulus, vertebral endplates and other disc structures must be undamaged.

Presently, most disc surgeries involve partial removal of the nucleus pulposus (nuclectomy). Or the nucleus is removed along with the entire intervertebral disc (discectomy). Standard surgical tools, such as curettes, bone nibblers or pituitary rongeurs, and a variety of techniques have been adapted for these procedures. All of these prior art tools were designed for purposes other than spinal surgery and are poorly suited to nucleus removal, especially when other tissues must be spared from injury. Generally, surgeons have experience and training only for procedures that require incremental extraction of small pieces of the nucleus (micro or partial nuclectomy). When applied to complete nuclectomy these tools lack the flexibility and control to remove all of the nucleus and invariably cause damage to the surrounding annulus fibrosus and vertebral end plates. In addition, substantial skill and dexterity is required to produce satisfactory results. Even in the hands of an experienced surgeon, nucleus extraction can be the most prolonged and difficult stage of the newer forms of spinal surgery.

No devices or methods have been developed specifically to remove the entire nucleus while minimizing trauma to other tissues. Maintaining the integrity of surrounding tissue is necessary to hold the implant in place and allow proper support and separation of the surrounding vertebrae. Some the implants will function poorly or risk new herniation if 20% or even as little as 10% of the original nucleus is left behind. A clean bed, free of nuclear material in critical locations, within which to deploy or graft the implants will also be crucial to the success of surgery. As a result, special methods, tools, or procedures are needed that can cleanly remove the nucleus without damaging the fibers of the annulus.

In an effort to address some of these limitations, physicians and researchers are searching for new methods of treatment for the herniated nucleus pulposus. They are looking at treatments that restore the function of the nucleus, regenerate the structure of the annulus, or are implanting artificial discs. Each of these proposed treatments introduces new difficulties and will need additional support mechanisms to prepare for the procedures. One of the most promising therapies is nucleus replacement. It is superior to traditional disc fusion because it restores movement and function to the disc space. It also promises to be superior to artificial disc implantation because much more of the original tissue is preserved, the procedure is faster, and there is less risk of malpositioning. Neither fusion nor artificial disc implantation are likely to ever be compatible with percutaneous access and thus carry a greater risk of infection and damage to other tissues or organs.

Most approaches to nucleus replacement will require removing the entire nucleus. Currently, there are few methods of removing the nucleus to prepare for nucleus replacement. These include the use of manual surgical implements such as curettes, bone nibblers, and pituitary rongeurs. The procedure involves incremental extraction of small pieces of the damaged portion until a the surgeon judges that a sufficient amount has been removed.

There are few companies currently looking at methods for removal of the nucleus pulposus, as nucleus replacement is a fairly new treatment modality. Clarus Medical has developed the ‘cut and suction’ method of percutaneous discectomy. Their product is the Nucleotome, a mechanical device with a blunt drill passing through a cannula that enters the disc site. It uses a rounded tip, shaped like a blunt drill to decrease the risk of cutting into the annulus. Stryker Corporation offers another rigid design, the “Dekompressor”, a percutaneous discectomy probe. It has a battery-operated disposable hand piece attached to a helical probe. The cannula allows access to the disc space, and the probe rotates and removes nucleus material through a suction mechanism. Both devices are too stiff to easily remove all of the nucleus

ArthroCare Corporation, has worked on coblation technology, which involves the use of low energy radio-frequency waves. This energy creates an ionic plasma field from the sodium atoms found in the nucleus. A molecular dissociation process occurs due to this low temperature plasma field, which converts this tissue into gases that exit the treatment site. The product is named the Spine Wand. It acts as drill as it is advanced into the disc. The tissue is converted into gas that exits the disc through the cannula. An accessory to the Spinal Wand is the System 2000 Controller. This accessory uses a combination of ablation, resection, coagulation and suction. A bipolar cautery is employed. However, the insertion depth up to the annulus must be predetermined and the wand is difficult to steer to remote parts of the nucleus space.

Laser discectomy employs laser energy to vaporize portions of a diseased disc. It is compatible with through minimally invasive surgery. However, laser techniques are generally useful to remove only small amounts of material because of the heat generated and other limitations. In addition, vaporized material expands to a gaseous phase and must be removed.

This invention proposes devices and methods directed to improving complete removal of the disc nucleus. The new process must be a relatively quick and cost effective alternative to current procedures. In addition, the new method or device must facilitate a complete and clean removal of the disc in a safe manner that does not compromise the integrity of the annulus.

### OBJECTS OF THE INVENTION

An object of the present invention is to overcome the drawbacks described above and other limitations in existing systems by providing a surgical device to remove almost the entire nucleus from a spinal intervertebral disc.

Another object of the invention is to remove nucleus material with minimal or no damage to surrounding tissues or structures such as the disc annulus, vertebral endplates, spinal nerves or blood vessels.

Another object of the invention is to be minimally invasive and carry a low risk of infection or discomfort to the patient.

Another object of the invention is to aid imaging, by x-ray or other means, of the nuclear space and surrounding structures.

Another object of the invention is to provide a system and method that removes the nucleus rapidly.

Another object of the invention is to provide a system and method that allows a surgeon to remove the nucleus without prolonged training, practice or skill.

Another object of the invention is to provide a system and method that removes the nucleus while allowing the surgeon fine control of the procedure.

These and other objects of the invention are accomplished according to various embodiments of the invention.

### BRIEF DESCRIPTION OF THE DRAWINGS

FIG. 1 is a frontal-lateral view of the anatomy of a section of the human lumbar spine.

FIG. 2 is a superior view cross section of the anatomy of a human lumbar intervertebral disc.

FIG. 3 is a superior view in cross section of a herniated human intervertebral disc.

FIG. 4 is a side view representation of the human spine in the vicinity of a herniated disc.

FIGS. 5A and 5B show a multiple port suction embodiment of the present invention.

FIG. 6 shows another multiple port suction embodiment of the present invention.

FIG. 7 shows yet another multiple port suction embodiment of the present invention.

FIG. 8 shows a multiple port suction embodiment of the present invention with protrusion features.

FIG. 9 shows another view of the multiple port suction embodiment of the present invention with protrusion features.

FIG. 10 shows a side view of a pinching embodiment of the present invention.

FIGS. 11A and 11B show a side views of a multiple arm pinching embodiment of the present invention.

FIG. 12 shows a side view of a multiple-vane collector embodiment of the present invention located in the nucleus space.

FIG. 13 shows a closer view of the vane collector embodiment of FIG. 12.

FIG. 14 shows a reciprocating and articulating plunger embodiment of the present invention.

FIG. 15 shows a rotatable, multiple-vane embodiment of the present invention.

FIG. 16 shows a rotatable an alternate multiple-vane embodiment of the invention in FIG. 15.

FIG. 17 shows a scissor arm embodiment of the present invention.

FIG. 18 shows a conveying embodiment of the present invention.

FIG. 19 shows a spiral conveying rod embodiment of the present invention.

FIG. 20 shows an expandable straining embodiment of the present invention driven by an inflatable member.

FIG. 21 shows the embodiment in FIG. 20 with arm elements.

FIGS. 22A and 22B show a spiraling plow and suction embodiment of the present invention.

FIG. 23 shows an inflatable member and suction embodiment of the present invention.

FIG. 24 shows a second inflatable member and suction embodiment of the present invention.

FIG. 25 shows a third inflatable member embodiment of the present invention.

FIG. 26 illustrates deployment of a deployable straining and suction embodiment of the present invention.

FIG. 27 shows combined inflatable and directional suction members as an embodiment of the present invention.

FIG. 28 shows the inclusion of further elements to the embodiment in FIG. 27.

FIG. 29 shows a detail of one embodiment of the distal portion of the suction member of FIG. 28.

FIG. 30 shows a further detail of one embodiment of the distal portion of the suction member of FIG. 28.

FIG. 31 shows a another view of the distal portion of the suction member of FIG. 30.

FIG. 32 shows another detail and element of the distal portion of the suction member of FIG. 29.

FIGS. 33A, 33B and 33C show a positioning device to aid deployment of the various embodiments of this invention.

FIG. 34 shows an oscillating member and suction embodiment of the present invention.

FIG. 35 shows the embodiment of FIG. 34 with multiple oscillating members.

FIG. 36 shows a distal translational motion control mechanism for use in the present invention.

FIG. 37 shows another embodiment of motion control with suction elements.

FIGS. 38A and 38C show a plowing vane embodiment of the invention in perspective and cross-section views.

FIG. 38B shows an alternate embodiment of the plowing vane.

### DETAILED DESCRIPTION OF THE INVENTION

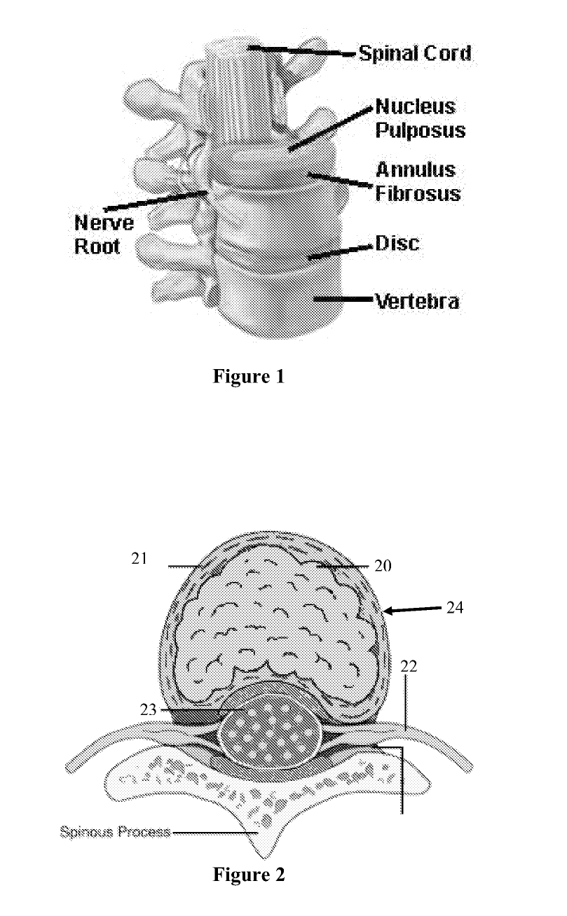

This invention overcomes various limitations of prior art means to remove nucleus pulposus from spinal intervertebral discs. FIG. 1 shows a section of the lumbar spine with major anatomic features labeled. Vertebrae are the bones that provide essential strength and stiffness to the spine and afford protection to the spinal cord, spinal nerve roots and major blood vessels (the blood vessels are not shown but are located opposite the spinal cord). The discs located between vertebra provide the spine with the ability to articulate by lubricating and separating the vertebrae.

FIG. 2 is a superior sectional view through an intervertebral disc 24 of the lumbar spine, the front of the body is upward in this view. Spinal nerves 22 radiate from the spinal cord 23, located posterior to the spine, to provide control and sensation to various segments and organs of the body. The disc 24 is roughly kidney shaped and defined by the annulus fibrosus 21. The annulus is composed of concentric layers of fibrous tissue that seal the space between vertebra located above and below the disc (not shown). Each layer of annulus 21 connective tissue is comprised of type I collagen oriented at approximately 30°. Successive annulus 21 layers alternate the 30° angle to provide substantial resistance to pressure from inside the disc 24. Within the space defined by the annulus 21 is the nucleus pulposus 20. The nucleus is avascular and comprised of hydrated mucoprotein gel and type II collagen fibers.

The intervertebral disc functions somewhat like a water bed to allow articulation of the spine. When a person is upright substantial hydrostatic pressure is developed within the disc 24 and this pressure increases at lower portions of the spine, particularly the lumbar and sacral region. The annulus 21 serves to contain nucleus 20 that is under pressures in the range of 690 to 2000 kPa (100 to 300 psi). Articulation of the spine is accommodated by displacement of nucleus material from one side of the nucleus space to another. In a normal, healthy spine the vertebrae are prevented from contacting each other even at maximal angles of articulation.

In young adults the intervertebral disc 24 is approximately 7 to 9 mm thick. With age and disease the hydration level of the nucleus 20 decreases. This thickens the nucleus from a soft gel-like consistency to become relatively stiff. Further degeneration with age and disease can occur to both the nucleus 20 and the annulus 21. This may allow the thickness of the disc 24 to decrease until, in the final stages, the vertebrae are in contact during some or all postures and movement. Contact between vertebrae damages these bony structures and generates substantial pain. Disc thickness greater than approximately 4 mm is presently considered suitable for nucleus replacement therapy. At lesser thickness treatment will usually involve removal of the disc 24 for spinal fusion or implantation of an artificial disc.

Because the nucleus 20 is avascular there are no living cells and exchange of fluids is through the cartilaginous endplates (not shown) covering the vertebral body. The endplates are a thin layer of primarily hyaline cartilage. The endplates are important to proper function of the intervertebral disc. In traditional therapies of fusion and disc replacement the endplates are not preserved so surgical techniques generally disregarded protection of the endplates. With motion restoration implantation of nucleus replacements the endplates must be protected from damage.

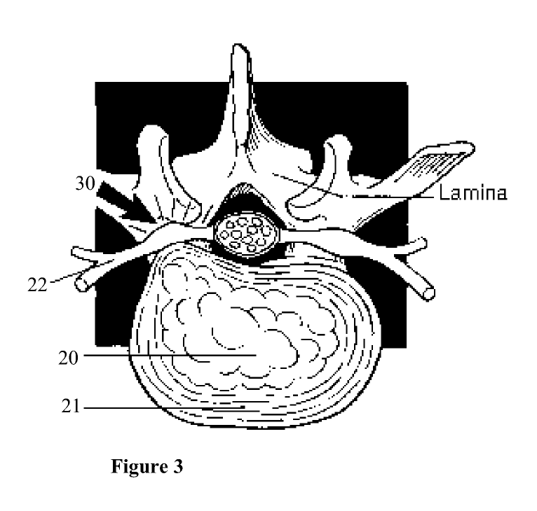

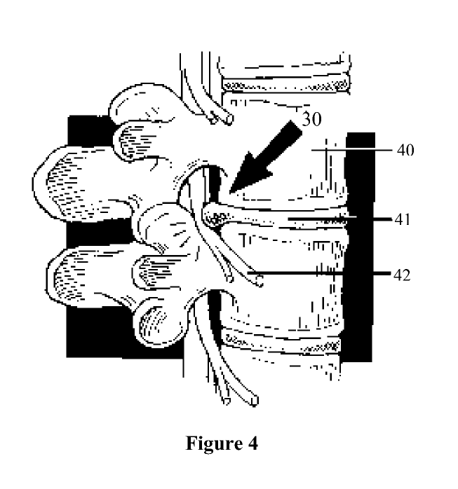

Similarly, with age and disease the annulus 21 may become weakened. This is a frequent cause of herniation, as illustrated in FIG. 3. As shown, the annulus 21 has weakened under pressure exerted by the nucleus 20 (in response to compression from the vertebrae) and compresses spinal nerve root 22. FIG. 4 is lateral view of a disc 41 herniation impacting spinal nerve 42 caused by annular failure 30. Similarly, the annulus 21 can fail such that nucleus material 20 exits the annulus and causes a direct effect on the nerve. In addition to being one of the major causes of disc therapy, degeneration of the annulus makes it vulnerable to damage during nucleus removal. The various embodiments of the present invention provide means of protecting the annulus from penetration or disruption.

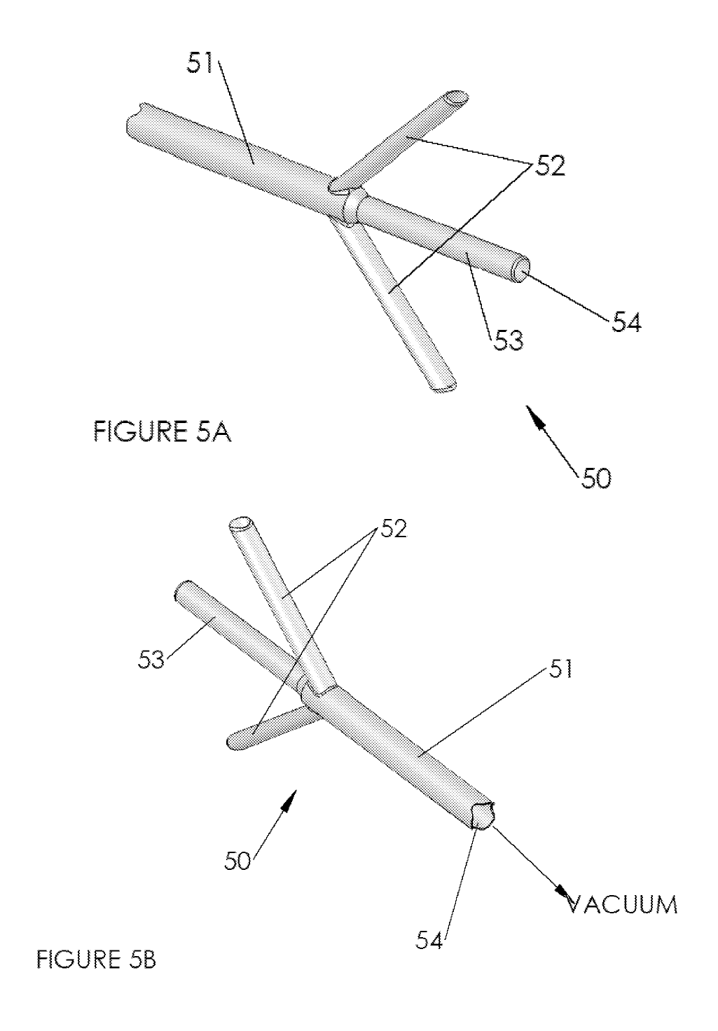

A first embodiment of the present invention 50 illustrated in FIGS. 5A and 5B contemplates a hollow tube 51 terminating at the distal end in a plurality of shorter tubes 52 and 53. Vacuum applied to the proximal end of tube 51 provides suction through lumen 54 at the opening of tubes 52 to remove nucleus 20 material. The hollow tube 51 preferably has a smaller cross-section area than the sum of cross-section areas of shorter tubes 52 and 53 yet has a larger cross section than any of the single tubes 52 or 53. The hollow tube 51 may be manipulated to move shorter tubes 52 through the nucleus space and remove substantially all of the nucleus material.

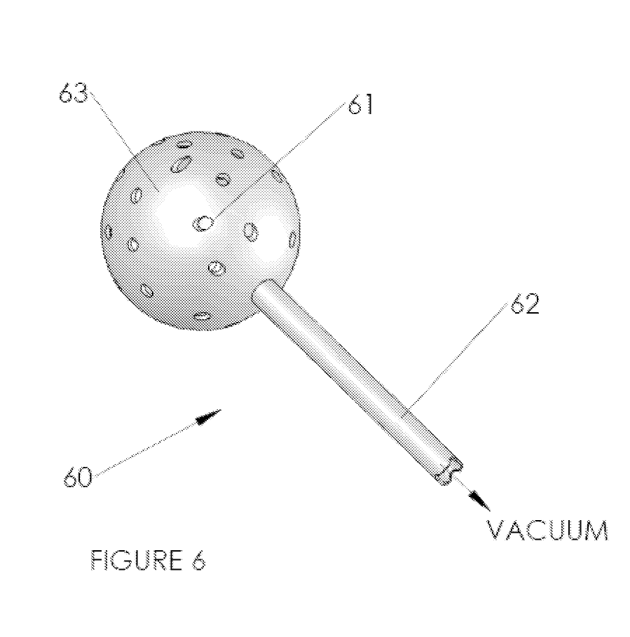

FIG. 6 shows another embodiment of the present invention 60 where hollow tube 62 terminates in a plurality of openings 61 in a roughly spherical plenum 63 with a diameter larger than the diameter of tube 62. Vacuum applied to the proximal end of tube 62 provides suction at each of the openings 61 to remove nucleus 20 material. An advantage of the present embodiment 60 is that the spherical conformation of the plenum 63 serves in preventing injury to the annulus.

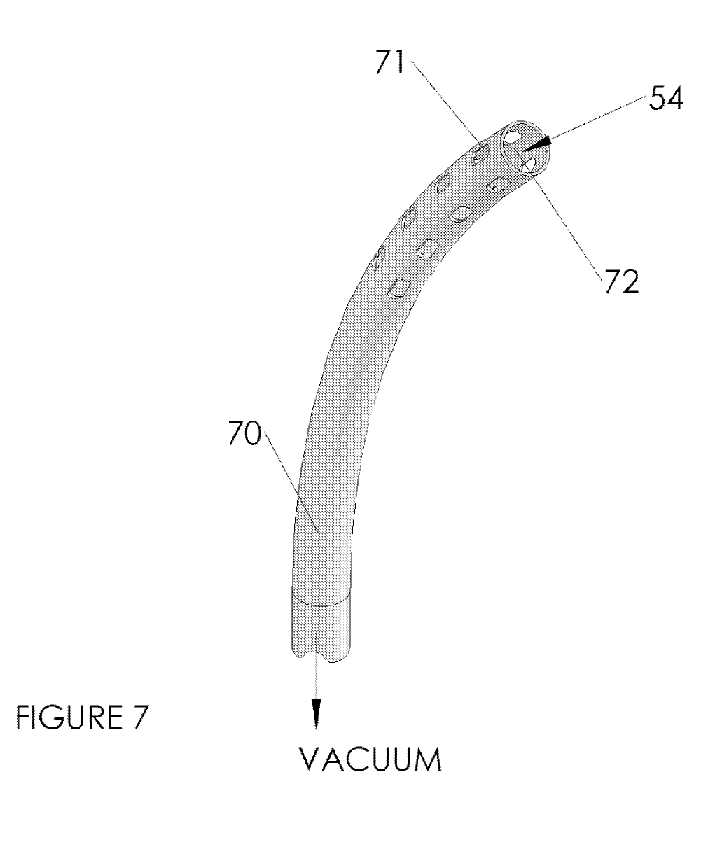

FIG. 7 shows another embodiment comprising a hollow tube 70 providing suction to distal side openings 71 and distal tip opening 72 when a vacuum is applied to a proximal end of tube 70. The illustrated distal portion of tube 70 is navigated throughout the nucleus 20 space to remove nucleus material. Tube 70 preferably has a terminal radius approximately the same as the inner radius of the annulus 21 of a human intervertebral disc.

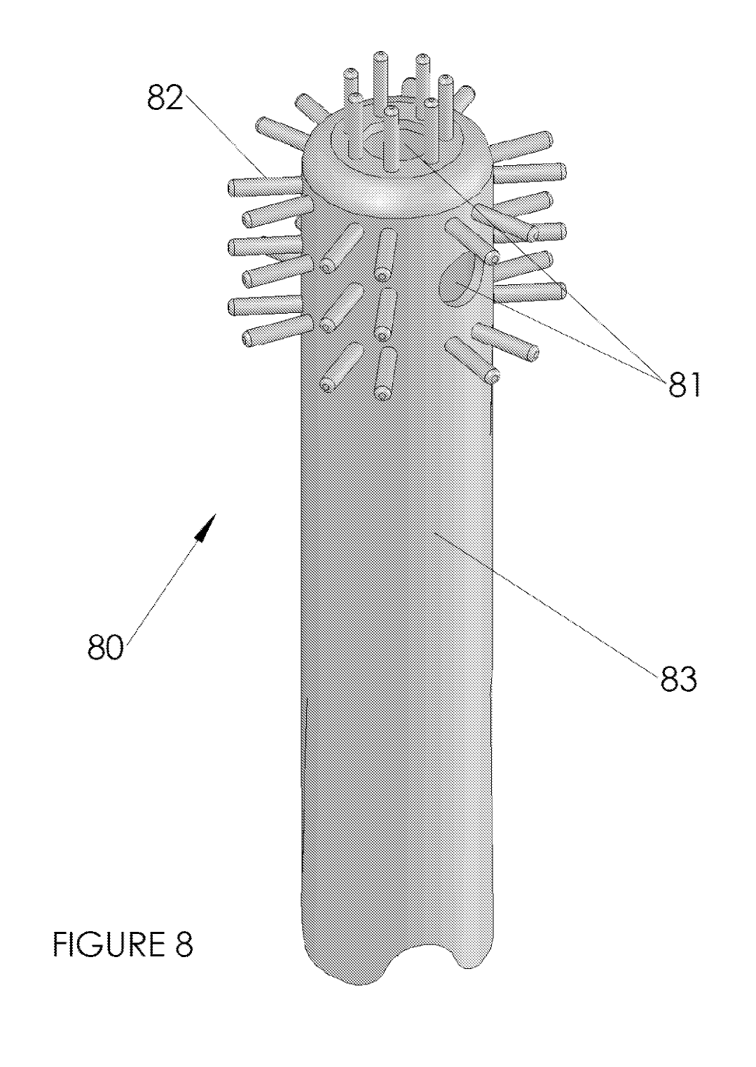

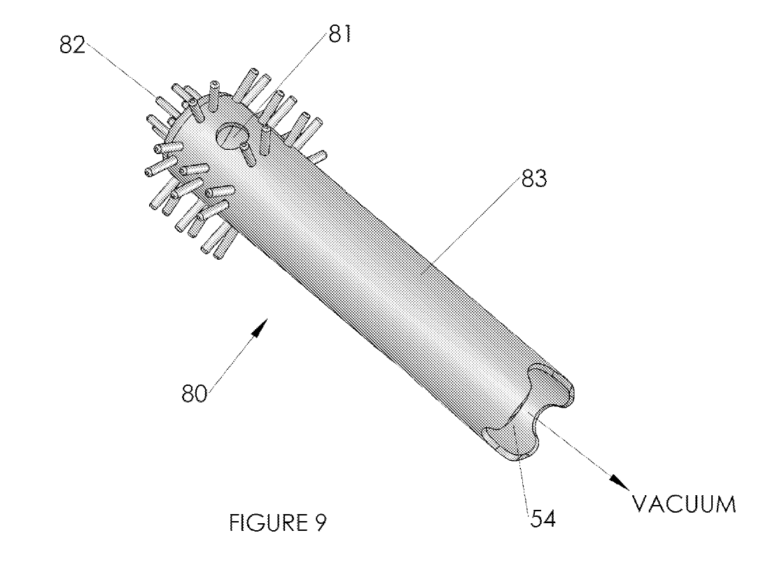

FIG. 8 shows an embodiment of the present invention 80 comprising a hollow tube 83 employing suction through openings 81 located on the distal side and tip. Features 82 are in the shape of fibers or short ridges that can be employed to disrupt the nucleus 20 material as the tube 83 is moved through the nucleus space. FIG. 9 is a proximal side view of embodiment 80 comprising illustrating the lumen 54 in tube 83 through which suction is applied to the openings 81.

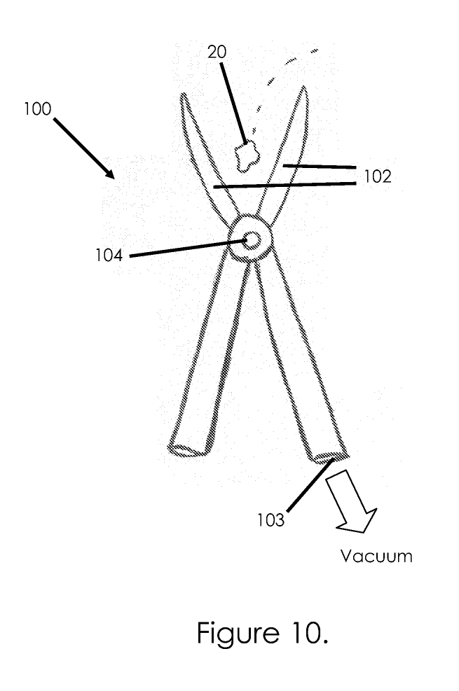

FIG. 10 illustrates a further embodiment of the present invention 100 comprising a hollow tube 103 terminating at the distal end in a grasping mechanism. The grasping mechanism comprises arms 102 that may be opened and closed by pivoting about pin 104 when activated by mechanisms operated at the proximal end of tube 103 (not shown). The grasping mechanism serves to liberate pieces of nucleus material 20 which are then removed from the nucleus space through tube 103 by suction or carried out of the disc space by removing the mechanism 100 with the arms 103 together.

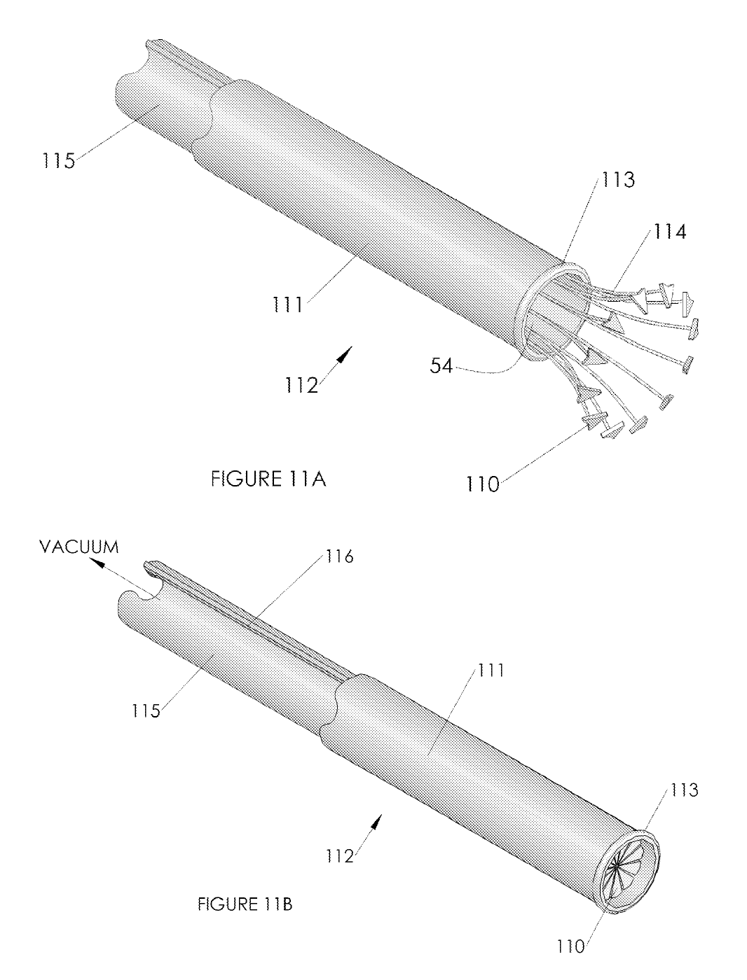

The embodiment of the present invention 112 shown in FIGS. 11A and 11B is comprised of an outer tube 111 containing a plurality of extensible tips 110 at the end of rods 114. The rods are threaded through at least part of the length of tube 111 and attached to inner tube 115 so that as inner tube 115 is controllably advanced from the proximal end the tips are moved away from the end of tube 111. Spring force in rods 114 cause the tips to move apart when advanced while ring apparatus 113 serves to define the point at which the diverging spring force is constrained. A cycle of advancing the rods into nucleus material and retracting them causes pieces of the nucleus material to be brought into proximity with the distal opening of tube 111. The tube 111 may removed from the nucleus space and each piece of nucleus material discarded or a vacuum may be applied to the proximal end of tube 111 through lumen 54 to remove nucleus 20 by suction. An optional guide ridge on the exterior of inner tube 115 matches a channel (not shown) on the inside of outer tube 111 to limit rotation and assure positioning of inner tube 116 within the outer tube 111.

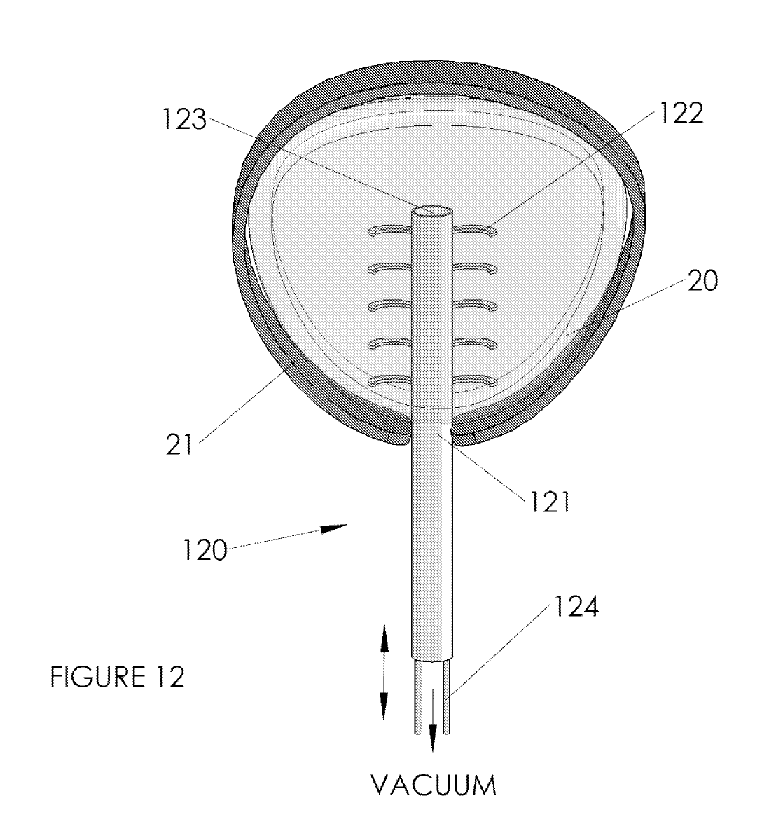

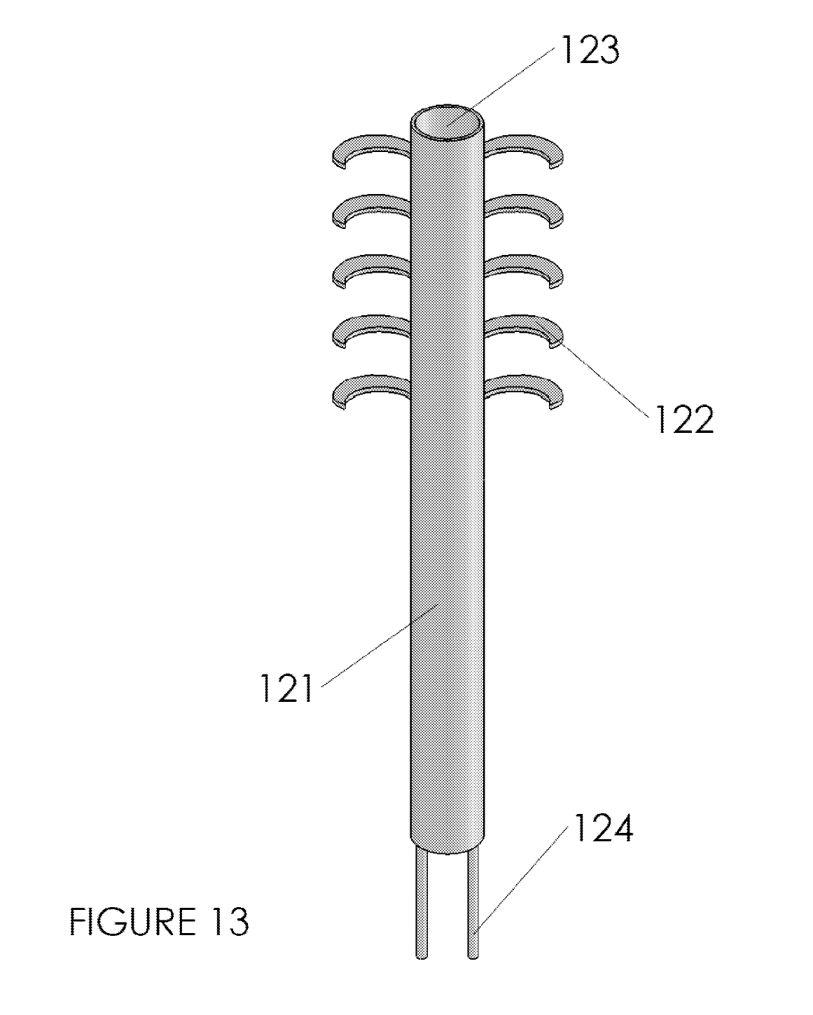

FIGS. 12 and 13 show another embodiment 120 of the present invention that comprises a hollow tube 121 with a distal opening 123 and a plurality of partially curled circumferential ridges 122. The ridges may be moved with one or more control rods 124 from a position substantially perpendicular to the tube 120 to an angle of approximately 30° to 45° (not shown). The ridges are preferably softer than the annulus to prevent injury to the annulus but disrupt the softer nucleus material and allow pieces of nucleus to be removed by suction through distal tip opening 130 or side openings (not shown), or entrapped and removed when the device is withdrawn from the nucleus space.

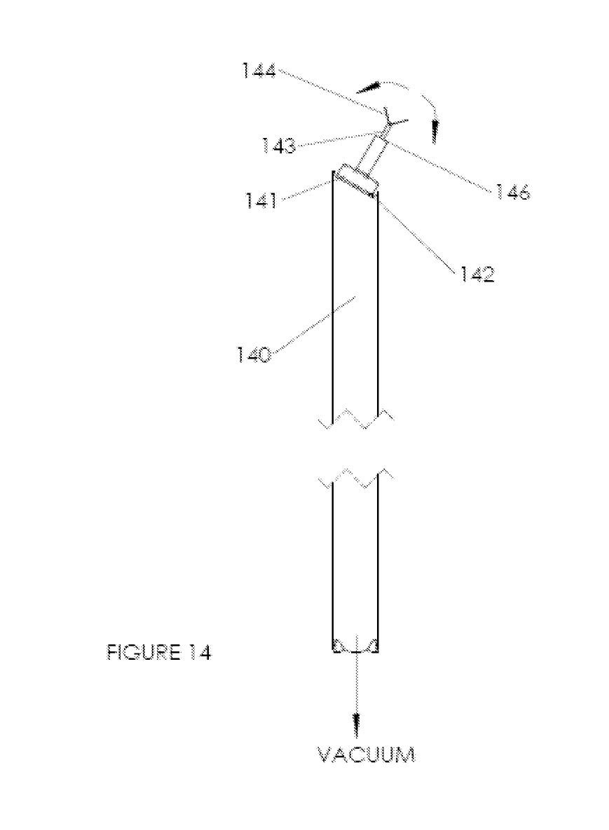

In a further embodiment of the present invention, the distal portion of a reciprocating apparatus is shown in FIG. 14. Hollow tube 140 comprises a collar 141 attached to the tube 140 by angleable joint 142 which further comprises a distal opening 146 that allows a vacuum applied to the proximal end of tube 140 (not shown) to produce suction at opening 146. Rod 143 passes through opening 146 and can be reciprocally advanced and retracted. One or more blades 144 are attached to the distal tip of rod 143 and are used to bring pieces of nucleus material into proximity with the opening 146 to be removed by the suction. Further, the blades 144 may be flexed in a manner shown by arrows 145 to increase mobilization of nucleus material. Joint 142 enables the collar 141 to be directed in various directions to reach each portion of the nucleus space.

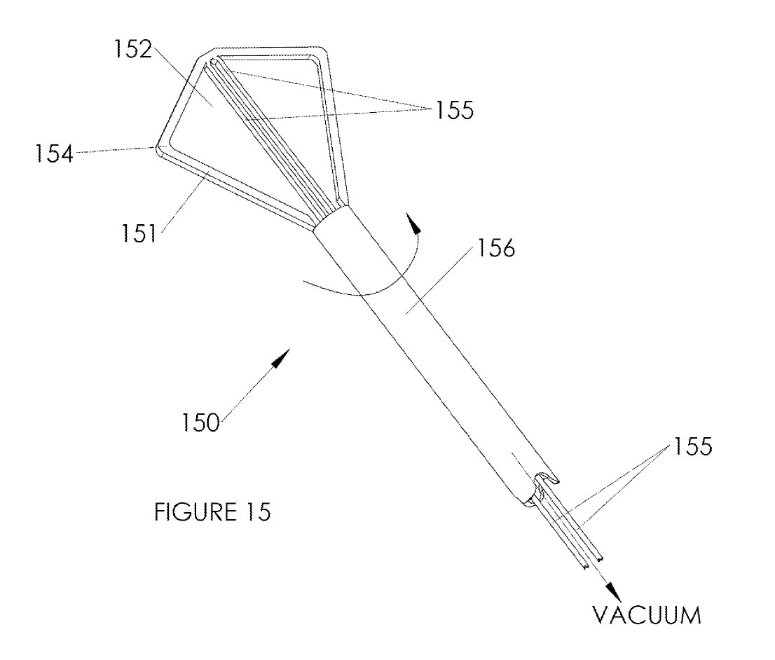

FIG. 15 shows an embodiment 150 of the invention that employs a hollow tube 156 to support and multiple arms that disrupt the nucleus when the device 150 is rotated. Each arm is comprised of two or more segments 151 and 152. First arm 151 is attached at one end to the tube 150 by a second pin joint or a flexural hinge. The second end of arm 151 is attached to arm 152 at flexural hinge 154, while the second end of arm 152 is attached at the distal tip of the apparatus to other arms 152. Control arms 155 can be extended longitudinally to expand arms 151 and 152. Nucleus material dislodged by motion of the arms may be extracted by suction through tube 150.

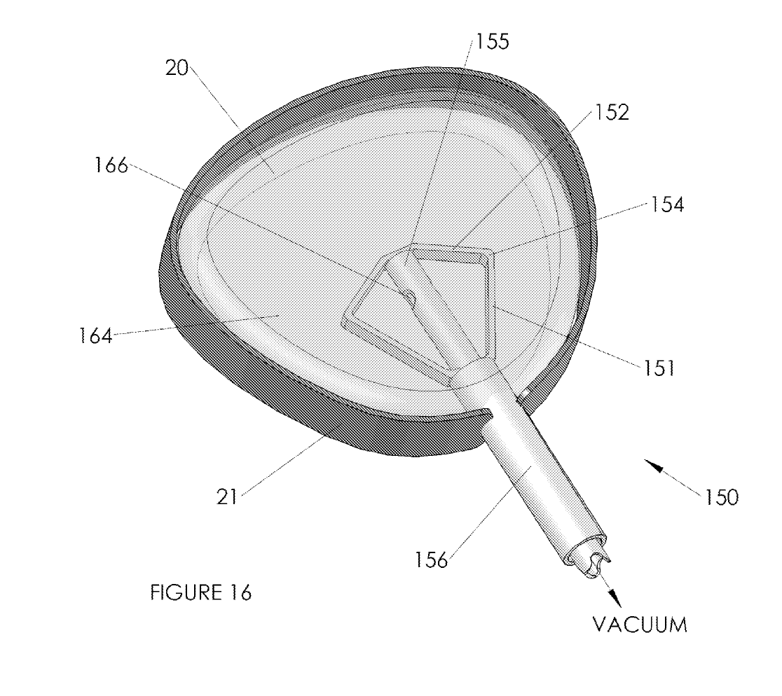

Deployment of the present embodiment 150 into the nucleus 20 space defined by the annulus 21 is portrayed in FIG. 16. Also portrayed in FIG. 16 is a particular embodiment with an inner hollow tube 155 used in place of the control arms in FIG. 15. Inner tube 155 contains one or more openings 166 to remove nucleus 20 material by suction.

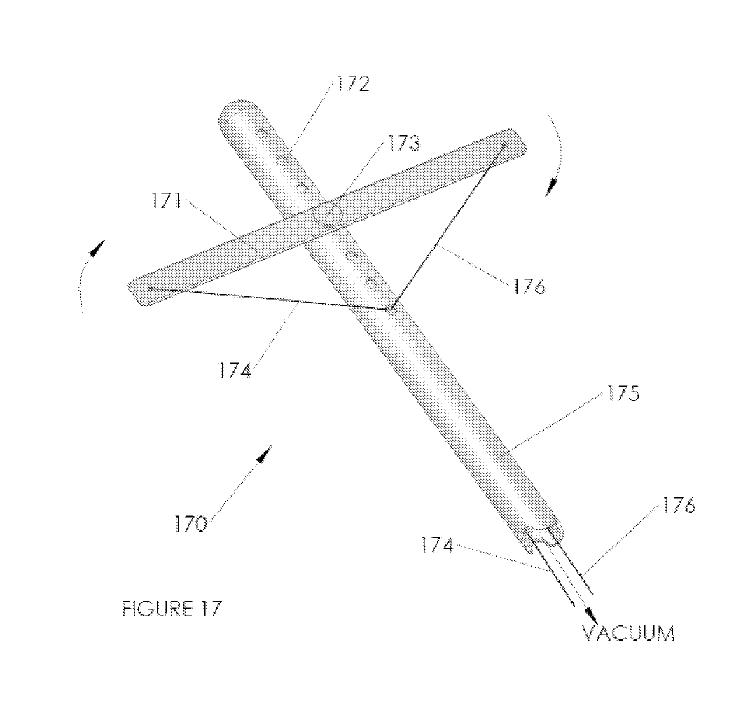

FIG. 17 shows a scissoring arm embodiment 170 of the present invention. Arm 171 is attached to hollow tube 175 at a pin joint 173. Tethers 174 and 176 are operated to rotate arm 171 from a position perpendicular to a parallel position with respect to tube 175 and disrupt nucleus material. In this view, rotation brings the nucleus material to a plurality of openings 172 in hollow tube 175 where the nucleus material may be removed from the nucleus space by suction.

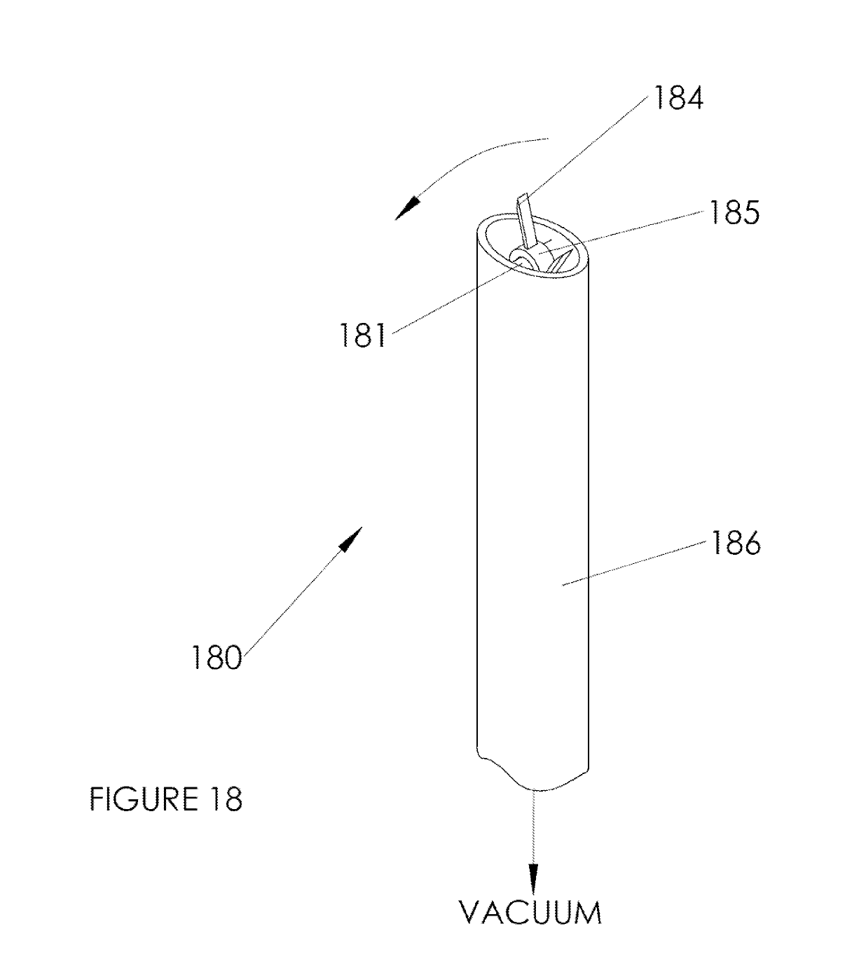

FIG. 18 shows a conveying embodiment 180 of the present invention. The conveying apparatus is attached to hollow tube 186 by connector 181 that allows the belt 185 of the conveying system to move through tube 186. A plurality of paddles 184 are attached to a belt 185 that may be guided in a loop in two direction, as indicated by the two-headed arrows 182. Nucleus 20 material are moved with the paddles 184 to an opening in the hollow tube for removal from the tube 186.

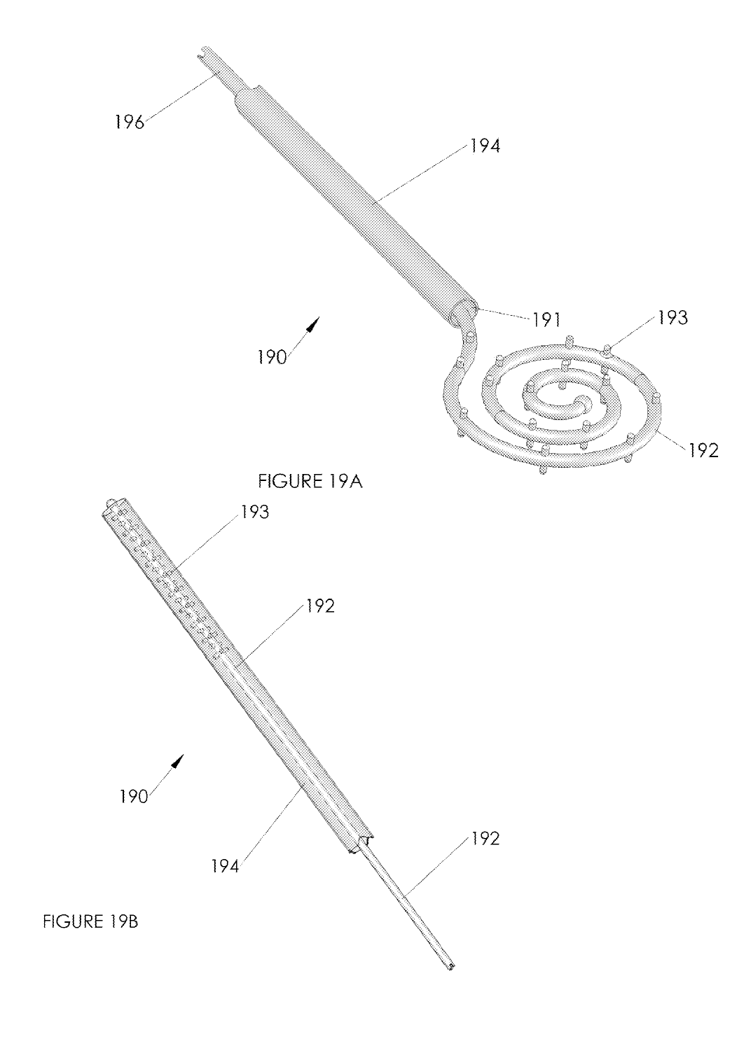

FIGS. 19A and 19B shows a spiral-formed apparatus 190 comprising a wire 192 that coils inward when extended from a hollow tube 194 through a distal opening 191. FIG. 19B shows the wire 192 retracted into the tube 194. Fingers 193 on wire 192 capture nucleus material and carry it through the tube 194. Vacuum applied to the tube 194 may be employed to aid removal of nucleus 20 material by suction. Tube 194 is manipulated by advancing and retraction and changing the angle of the tube 194 to reach substantially all of the nucleus space. A rigid rod 196 may be attached at the proximal end of the wire 192 to control deployment of the wire.

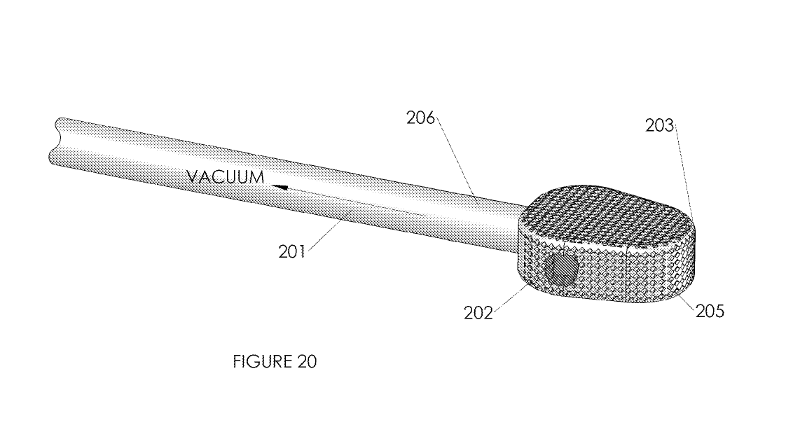

FIG. 20 shows a cutting balloon apparatus comprising an inflatable balloon 202 and cutting mesh 203 comprised of an elastic material containing a plurality of openings 205 attached to the distal end of tube 206. Tube 206 contains a plurality of lumens 201 (not shown) that are employed to inflate the balloon 204 and remove nucleus 20 material when controlled pressure and vacuum, respectively, are applied to separate lumens at the proximal end (not shown) of tube 206. When the balloon 202 is inflated the cutting mesh (resembling a strainer or screen) 203 is forced through the nucleus space to disrupt nucleus material that may be removed by suction through a lumen of tube 206 or with the cutting balloon apparatus as the balloon is deflated.

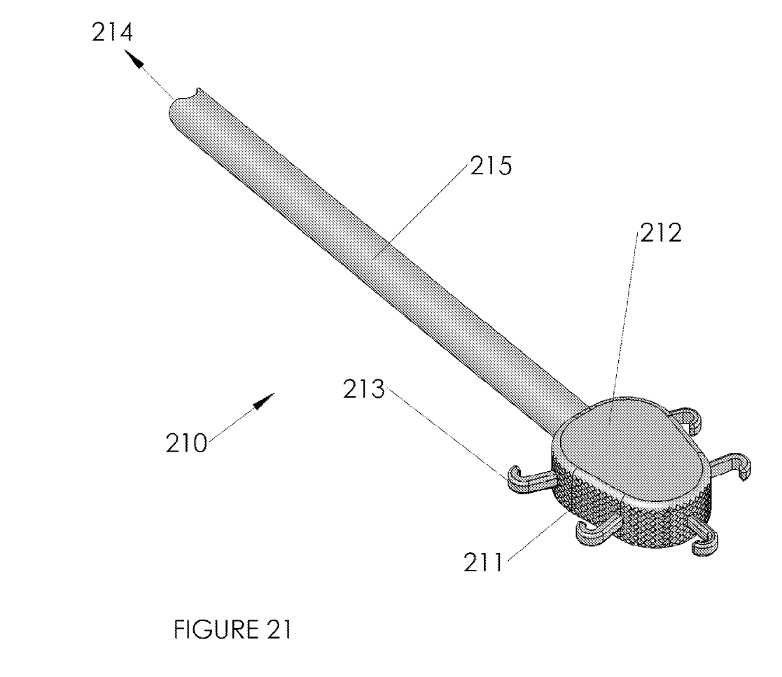

FIG. 21 is another embodiment 210 of the invention comprising a cutting mesh 211 that is expanded into the nucleus space defined by the annulus 21 by spring force as the mesh is advanced from the distal end of tube 210. The mesh 211 may incorporate hooks or other features 213 on the outside that further aid in disrupting the nucleus. As the mesh 211 is advanced through the nucleus space disrupted nucleus material passes through to the interior 212 of the mesh 211. Suction applied through hollow tube 215 removes nucleus material along the indicated path 214. The distal mesh and balloon combination may preferably provide a flat and smooth surface 212 that helps to prevent injury to the end plate tissue of vertebrae 40.

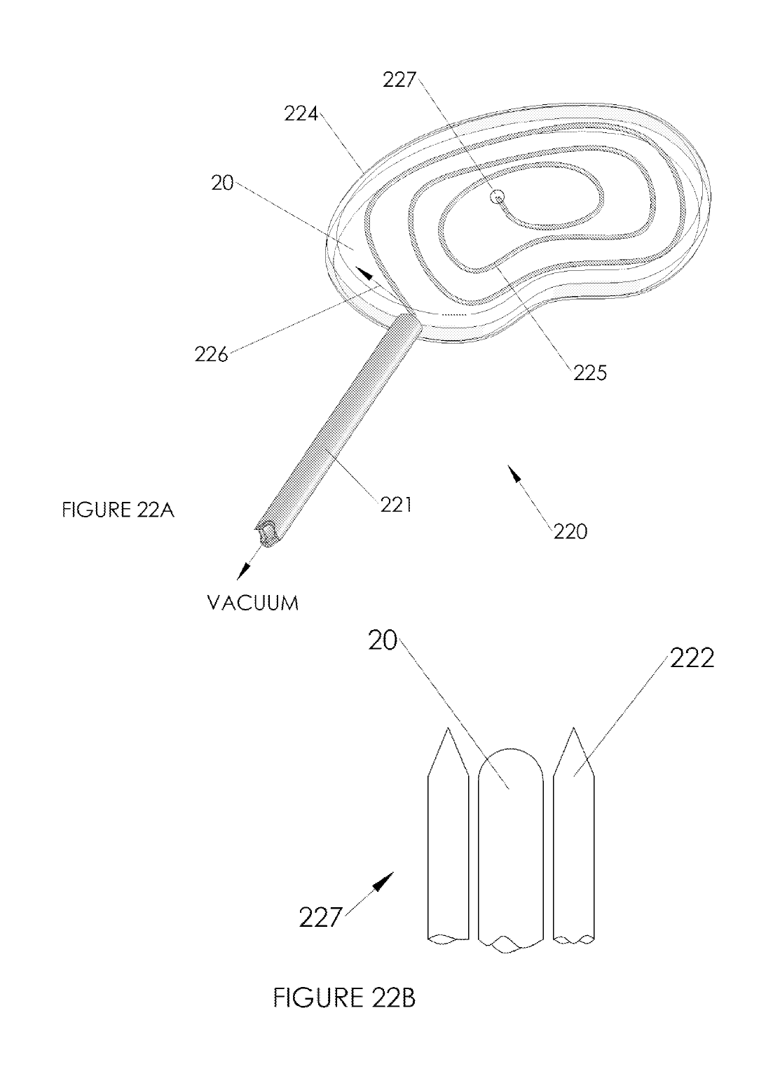

FIGS. 22A and 22B illustrate yet another embodiment 220 of the invention comprising insertion tube 221 that enters the nucleus 20 space through an opening in the annulus 21. A flexible tube 225 is advanced from the tube 221 and employs an outward spring force 226 to follow the inside edge 224 of the annulus 21 defining the nucleus space. When the flexible tube 225 has passed around the periphery of the nucleus space it begins to follow an inwardly spiraling path as more of the flexible tube is advance from the insertion tube 221 until the desired amount of nucleus material is removed. FIG. 22B is a detail of the distal portion of flexible tube 225. An approximately cylindrical scoop 222 is formed at the distal end of the flexible tube 225 that captures nucleus 20 material that is removed from the nucleus space by suction through flexible tube. The scoop 222 is comprised of soft material, preferably in the range of Shore A hardness 30 to 60, that prevents damage to the annulus.

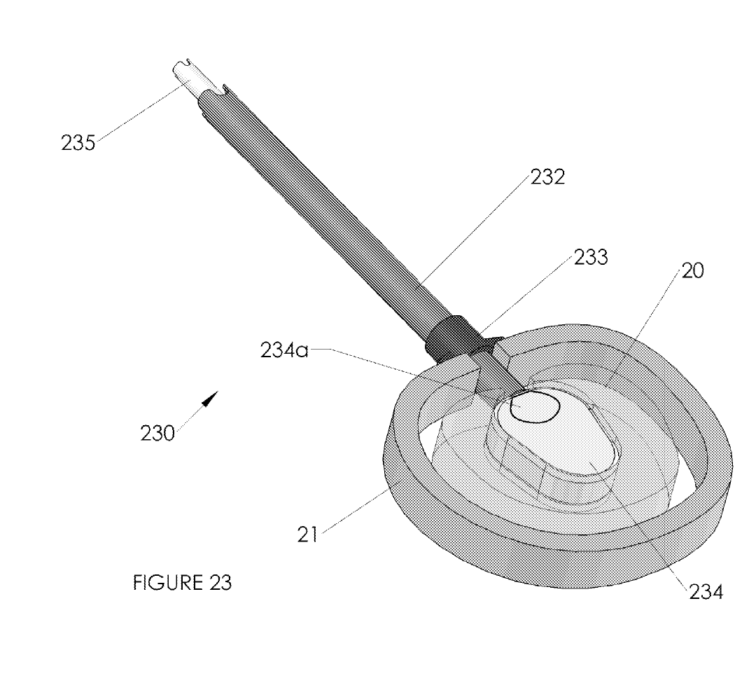

According to the embodiment of the invention illustrated in FIG. 23 tube 232 is placed within the nucleus space 20 of an intervertebral disc defined by annulus 21. A balloon 234 located at the end of the tube 232 is inflated with fluid from a collapsed shape 234a to progressively displace nucleus 20 material into a suction lumen 235 of tube 232 placed in communication with the nucleus space. Suction lumen of the tube 232 preferably has a vacuum or suction applied to its lumen at the proximal end and removes displaced nucleus entering the distal end from the body and prevents nucleus from exiting the disc and remaining inside the patient. The suction lumen 235 of tube 232 may incorporate a collar or other feature 233 that aids in sealing the opening in the annulus 21 to prevent escape of nucleus material and the balloon and allow a greater negative pressure to be developed. The physician or operator may manipulate the tube 232 by changing the angle that they enter the nucleus space and advancing or retracting the tubes within the nucleus space to navigate the geometry of the nucleus space so as to remove the desired quantity of nucleus.

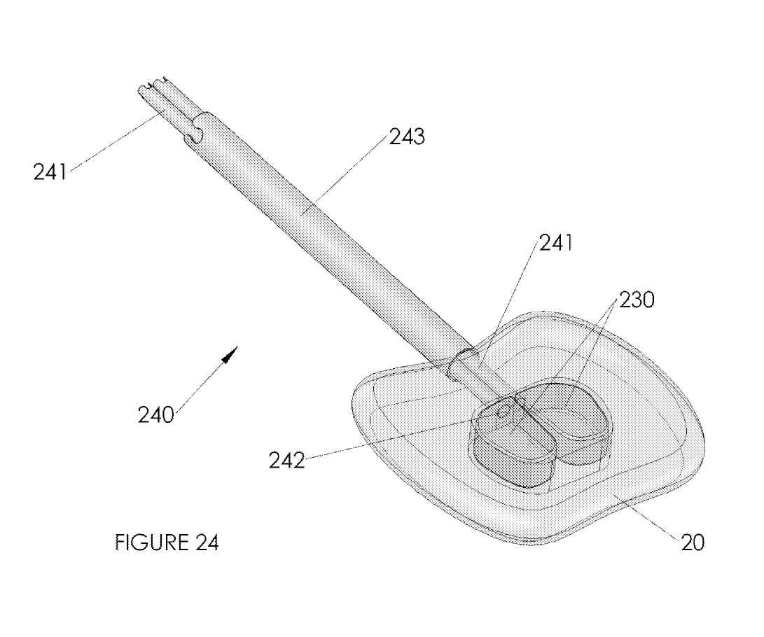

In a second embodiment of the invention 240, illustrated in FIG. 24, one or more balloons 230, or a single donut-shaped balloon is attached to the end of a first tube 243 containing lumen 241 that collects nucleus 20 displaced by the balloon 230. A vacuum may applied to the proximal end of lumen 241 to aid in removal of nucleus 20 through one or more openings 242.

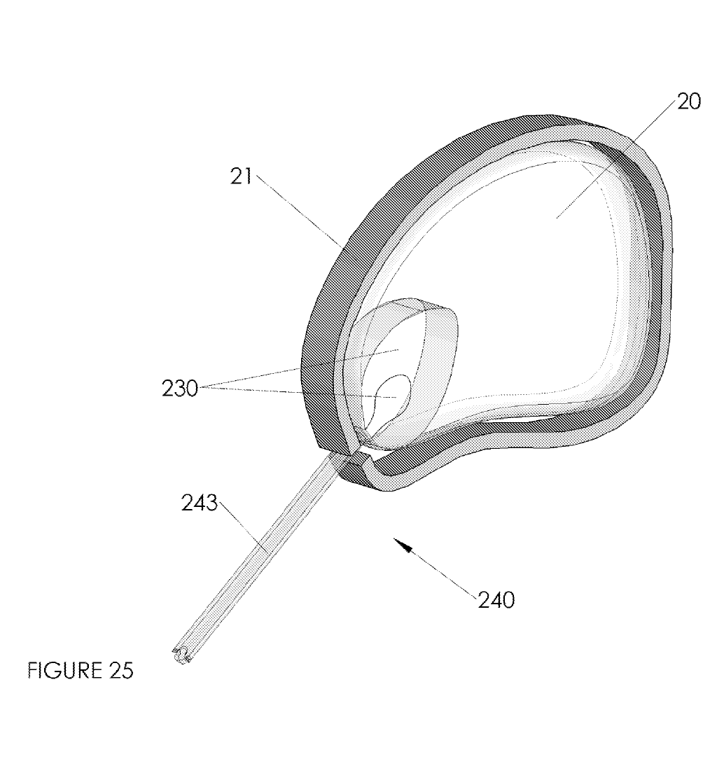

An example of deployment of a single balloon 230 from a tube 243 within the kidney-shaped nucleus 20 space defined by annulus 21 is illustrated in FIG. 25. Balloon 230 may be partially inflated and deflated one or more times to progressive mobilize nucleus 20 into the tube 243 or otherwise out of the disc. Tube 243 may be angled and repositioned one or more times in coordination with inflation of balloon 230 to optimize nucleus 20 removal. Balloon 230 is preferably inflated with an incompressible fluid having radio-opaque properties to aid visualization of the nucleus space and anatomy of the disc.

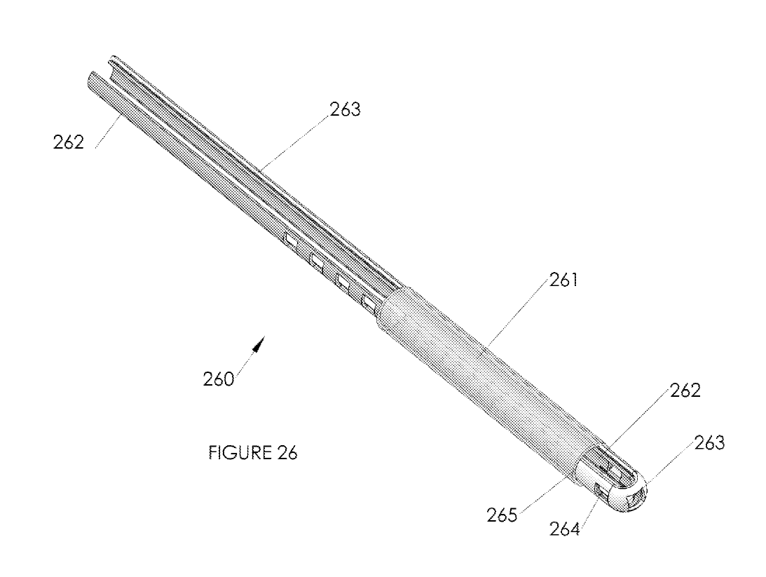

FIG. 26 shows deployment of an expand and capture apparatus 260. A one 262 or two-piece strap (262 and 263) is advanced into the nucleus space from tube 261 until it is in contact with the annulus circumscribing generally the entire nucleus space. The strap is comprised of a plurality of equally spaced openings each with a diameter of between 50% and 85% of the width of the strap. The strap preferably has a width approximately equal to the narrowest gap between vertebrae defining the sides of the nucleus space. Or, the width of the band may be between 2.5 and 5 mm and further comprise soft wipers or ridges to aid in forming a loose seal against the surfaces of the vertebrae. Alternatively, the strap 262 may be comprised of a plurality of hinged links each with an opening in the same range as described above. This embodiment of the strap resembles a bicycle chain. The chain-like band contains features at the link joints, such as tabs mating with slots, that constrain the strap to a generally convex shape as it is being advanced from the tube 261 (under compression) and provide for flexion in any direction when it is retracted.

As a strap 262 or 263 of apparatus 260 is advanced into the nucleus space nucleus 20 material is disrupted and forced through the openings in the strap. The width of the strap, or the wipers described above, ensure that essentially all of the nucleus material is forced through the openings in the strap and is prevented from escaping around the strap. Once a quantity of nucleus material is captured with the region defined by the strap the strap and be withdrawn into tube 261 carrying with it the entrained nucleus. Suction applied through the tube 261 can aid in removing material from the nucleus space 264. The strap may be repeatedly advanced and retracted until the desired quantity of nucleus material has been removed 263. The strap will preferably contain radiopaque material or features that help describe its outline and location when imaged by x-ray. When fully deployed the strap will aid in imaging the nucleus space.

Apparatus 260 may further comprise a second strap associated with the first strap. The second strap would have a width equal to or less than the first band and contain roughly the same number and size of openings as the first band. As the straps are advanced they are arranged so that the openings in both straps are aligned which allows nucleus material to pass through both straps. To prepare for retraction, the second strap is moved relative to the first strap sufficiently so that the openings in the two straps a no longer aligned and nucleus material is further disrupted and entrapped within the space defined by the straps. One or both straps 262 and 263 may be attached 265 at one end to the distal opening of tube 261.

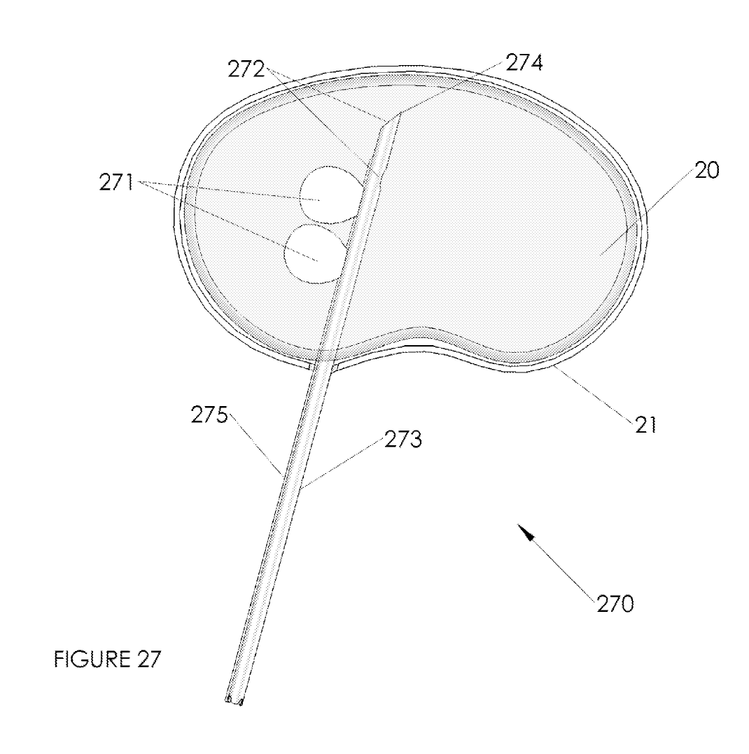

FIG. 27 illustrates a directional balloon and tube apparatus 270 of the present invention. One or more balloons 271 are attached to one side of the distal portion of a tube 273 containing a plurality of lumens 275 that provide proximal fluid communication to the balloons 271 and distal openings 272, and to respective pressure and vacuum sources at the proximal end (not shown) of tube 273. Openings 272 at the distal end and side of the tube allow nucleus 20 to be removed from the disc through one of the tube lumens. The tip 274 of tube 273 is made of a soft material, rounded or otherwise adapted to prevent damage to the annulus 21 as the tube is inserted and manipulated in the nucleus space.

By one preferred method, apparatus 270 is advanced into the nucleus space along the lateral wall defined by the annulus closest to the location that the apparatus penetrates the annulus (usually the location of annular failure in herniation). Suction is applied to the distal openings 272 through a lumen 275 while the apparatus 270 is advanced and throughout the nucleus extraction procedure. The apparatus 270 may be turned through 180° in alternate directions or 360° from its initial orientation so that nucleus 20 material to all sides is removed. At any time during the procedure the apparatus may be partially or completely retracted and re-advanced, with or without rotation, so that the distal openings 272 come into contact with a maximum of nucleus 20 material.

Once initial placement of the apparatus 270 is complete the apparatus is rotated to position the distal openings 272 toward the nucleus 20 space that still contains nucleus material. Suction continues on distal openings 272 while one or more balloons 271 are inflated to push the openings into contact with, and through, the nucleus material. During balloon 271 inflation the apparatus may continue to be manipulated by rotation and further advancement or retraction, as allowed by the position of the balloon, to bring the openings 272 into contact with remaining material and to navigate the apparatus through the nucleus 20 space. Inflation of balloon 271 also serves to displace nucleus 20 material around the tube 273 and into proximity with the openings 272 so that it can be removed from the nucleus space. Balloon 271 further contributes to removal of nucleus material by increasing the static pressure within the nucleus 20 space so that the net pressure across the openings 272 is higher relative to the applied vacuum. The process of balloon 271 inflation and manipulation of the apparatus continues until the desired quantity of nucleus material is removed.

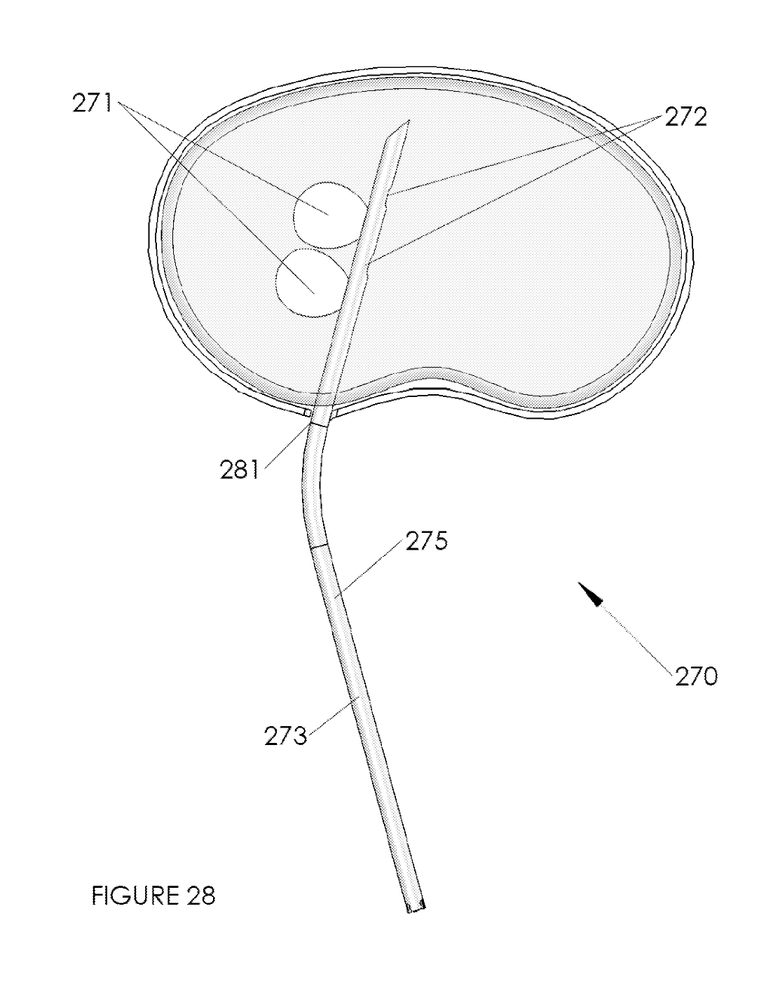

A further embodiment of the balloon and tube arrangement 270 is illustrated in FIG. 28. A plurality of openings 272 are formed on the side of the tube 273 opposite the balloon. Preferably, the number and size of openings define a longitudinal distance substantially equal to the co-linear dimension of the nucleus 20 space. It may also be advantageous to have openings 272 only on the side of tube 273 and not provide a distal opening. A further preferable configuration would permit certain openings 272 to be closed by advancing outer sheath 281 when they are not in contact with nucleus 20 material. This permits maximum vacuum pressure to be applied to the openings best able to remove nucleus.

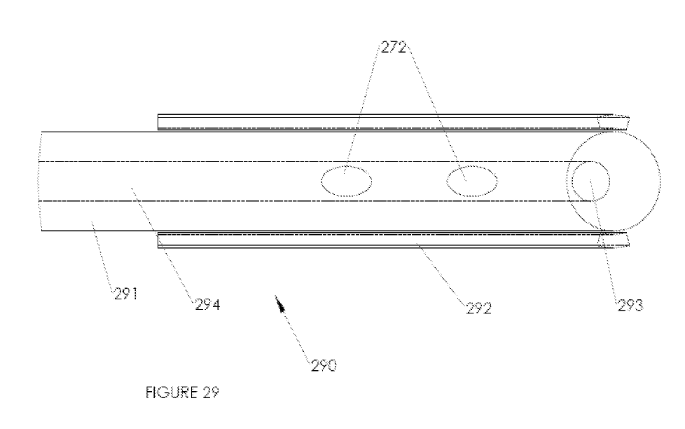

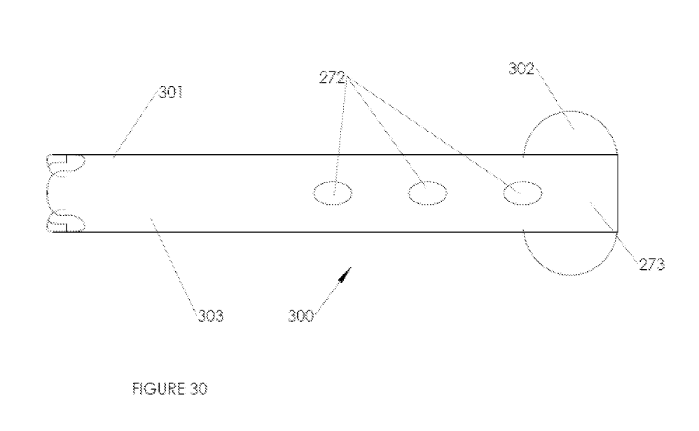

FIG. 29 shows details of a possible arrangement of the tip of a directional suction apparatus 290 that can be used alone or with the balloon and tube arrangement 270. Side openings 272 and distal opening 293 in tube 291 provide fluid communication between suction lumen 294 and nucleus 20 material outside the apparatus 290. Ridges 292 are a flexible material that conforms to the shape of the surrounding vertebrae and endplates to form a partial seal separating the two sides of the apparatus. When used in the balloon and tube arrangement 270 the ridges 292 aid in collecting nucleus 20 material to the openings 272 as the apparatus is moved through the nucleus 20 space. As well, ridges 292 aid in holding the balloon to one side of the tube 291 so that it does not interfere with movement of arrangement 270 or openings 272 and 293.

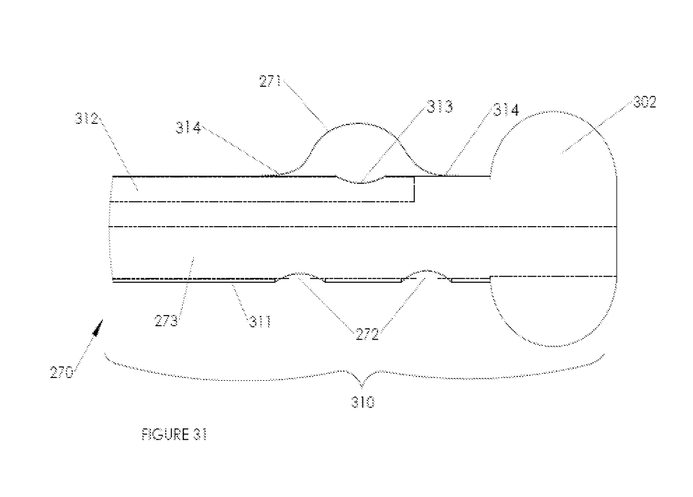

FIG. 30 shows one embodiment 300 of a soft tip 302 formed on tube 301. Tip 302 may be molded directly from tube 301 with a forming tool. Alternatively, tip 302 may be created from a different material, preferably with a lower Shore hardness than the material of tube 301, and attached to tube 301 with adhesive or heat / chemical welding. Tip 302 allows for distal opening 273 to communicate with lumen 303.

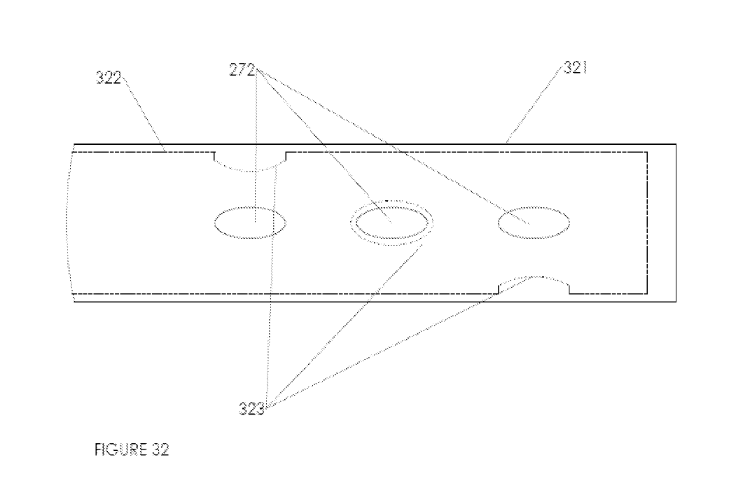

FIG. 31 presents a top view of a tip configuration 310 of balloon and tube arrangement 270. The tube 311 contains suction lumen 273 and inflation lumen 312. Side openings 272 communicate with suction lumen 273. Inflation opening 313 is located on the side of tube 311 opposite side openings 272 and communicates with inflation lumen 312. A balloon 271 is formed of a membrane sealed 314 to tube 311 around inflation opening 313. Fluid supplied under pressure to inflation lumen 312 passes out of inflation opening 313 and enters and enlarges balloon 271.

FIG. 32 illustrates the tip of a suction tube 321 that contains a second tube 322 that allows openings 272 in tube 321 to be selectively opened or closed. The second tube 322 has an outside diameter approximately equal to the inside diameter of suction tube 321 that provides a relatively close seal between the tubes 321, 322. Second side openings 323 in second tube 322 are preferably at least as large as the side openings 272 in tube 321. The second openings 323 are positioned at the same longitudinal positions as the side openings 272 but at different radial positions. Rotation of the second tube 322 within the suction tube 321 allows the side openings 272 to be selectively closed by orienting the associated second opening 323 away from the side opening 272. Various arrangements of second openings 323 within the second tube 322 may be made to provide different combinations of open and closed side openings 272 by rotation of second tube 322. A further use of second tube 322 is to cut nucleus 20 material that enters a side opening 272 into segments that aid removal of the nucleus material by suction.

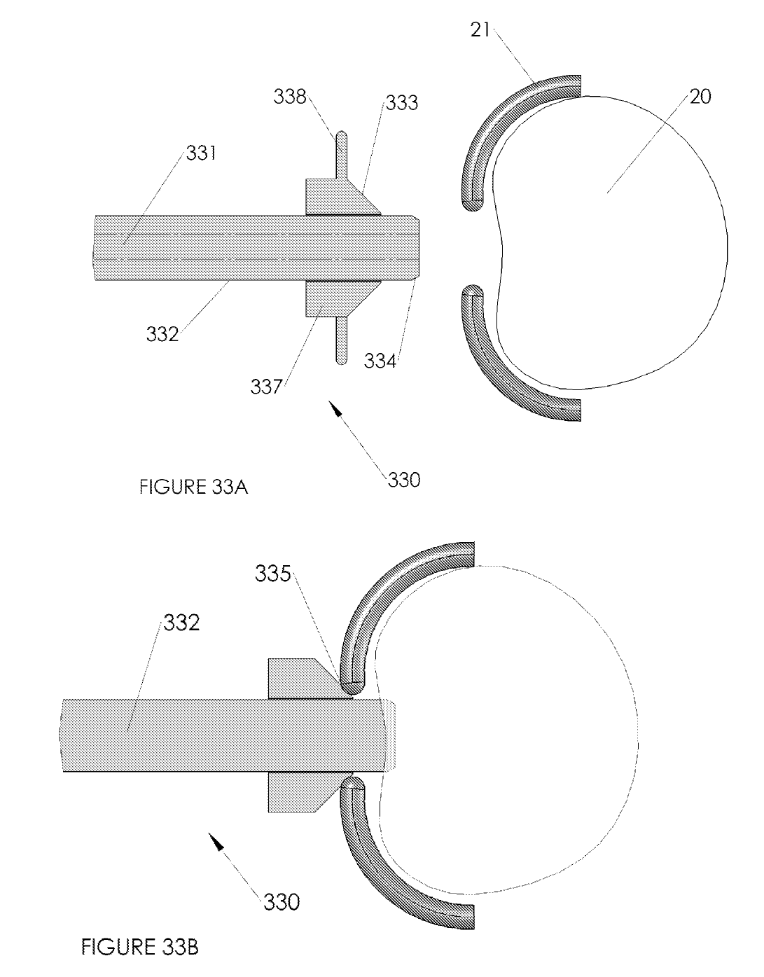

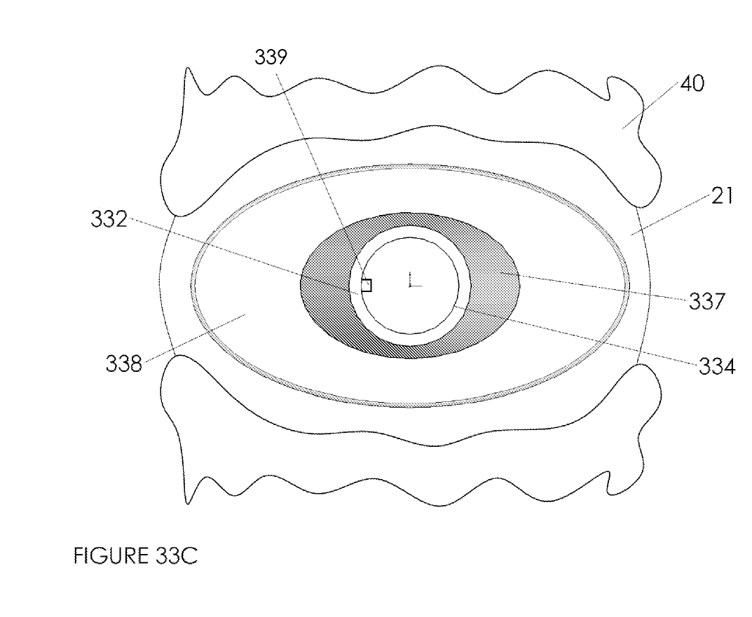

FIGS. 33A, 33B and 33C show three views of a sheath 330 to aid in inserting and positioning the various nucleus 20 removal embodiments of this invention. Tube 332 comprises a lumen 331, flange 337, tip 334 and flange extensions 338. The lumen 331 is sized to accommodate passage of a nucleus removal apparatus and guide it to the opening in the annulus 21. The sheath 330 protects the nucleus removal apparatus from damage and kinking as it is inserted and manipulated. Similarly, the sheath protects the annulus and other tissues from injury by the nucleus removal device. The distal tip 334 of the sheath 330 is tapered to ease insertion through an existing opening in the annulus 21. The tip 334 may also be comprised of a soft material and be further shaped to prevent injury to the annulus during insertion.

An important objective of the sheath 330 is to seal the opening in annulus 21 and prevent nucleus 20 or other materials from escaping the disc and being released into the body. Taper 333 on the flange 337 assists in providing a tight fit in the contact region 335 with the annulus 21. Further, the tapered or soft tip 334 will form a partial seal around the nucleus removal device. Flange 337 has an oblong shape defined by flange extensions 338 that allows a large contact area with the annulus 21 while fitting between the vertebrae 40. This shape of the flange 337 also keeps the sheath 330 oriented (rotation is prevented). A key 339 may be incorporated into the sheath so that a matching keyway on a nucleus removal device will serve to keep both devices oriented. Alternatively, markings on the proximal end of the sheath 330 (not shown) can be provided to indicate orientation.

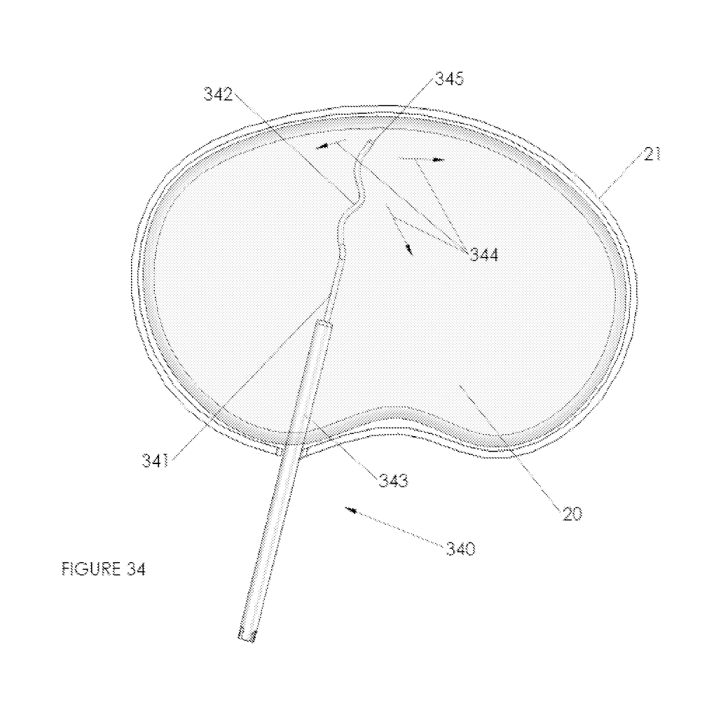

A further embodiment of a nucleus removal device 340 is illustrated in FIG. 34. It is comprised of a whip 342 located within the nucleus 20 space and attached to a vibration transmission rod 341. Vibrational motion is delivered to the rod 341 at the proximal end of the device 340 (not shown). The mechanical characteristics of the rod 341 are arranged so that the vibrational motion is transmitted efficiently from the proximal end to the distal end of the rod 341 with minimal actual motion within tube 343. Whip 342 has different mechanical characteristics that convert the vibrational motion transmitted through the rod 341 to substantial motion of the whip 341. The vibrational frequency and displacement delivered to the proximal end of the rod 341 is tuned to produce substantially more motion of the whip 342. Preferably, a standing wave motion 344 would be produced in the whip 342 by the vibrational motion. More preferably, the standing wave would only be present when the whip 342 is in contact with nucleus 20 material and would degenerate to smaller amplitude motion where the whip is in contact with annulus 21 or other material with different characteristics. The tip 345 of the whip 342 incorporates a button or other feature to limit injury to the annulus 21 or endplates. The tube 343 contains a lumen surrounding the rod 341 to which a vacuum may be applied at the proximal end for the purpose of removing nucleus 20 material by suction.

Vibrational motion 344 of the whip 342 of device 340 disrupts the structure of nucleus 20 so that it may be more easily removed by suction through tube 343. Tube 343 may be manipulated and whip 342 may be extended or retracted so that whip 342 can be directed to all parts of the nucleus 20 space. Tube 343 may also be advanced into nucleus space 20 to aid removal of nucleus material by suction.

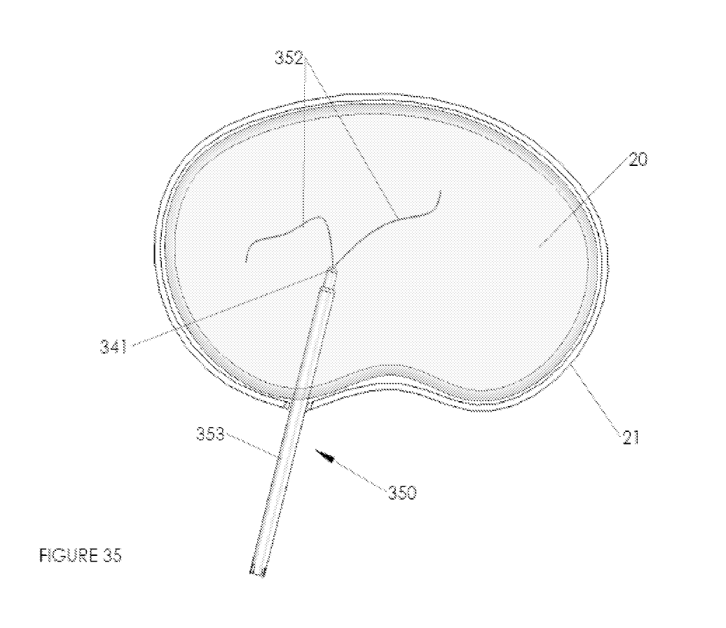

An alternate embodiment to a vibrational whip 350 is portrayed in FIG. 35. Instead of one long whip, as in the whip 342 in FIG. 34, there are two symmetrically configured whips 352 extending from the rod 341. In addition, rod 341 may be extended or retracted separately from tube 353 so that the whips 352 may be directed throughout the nucleus 20 space.

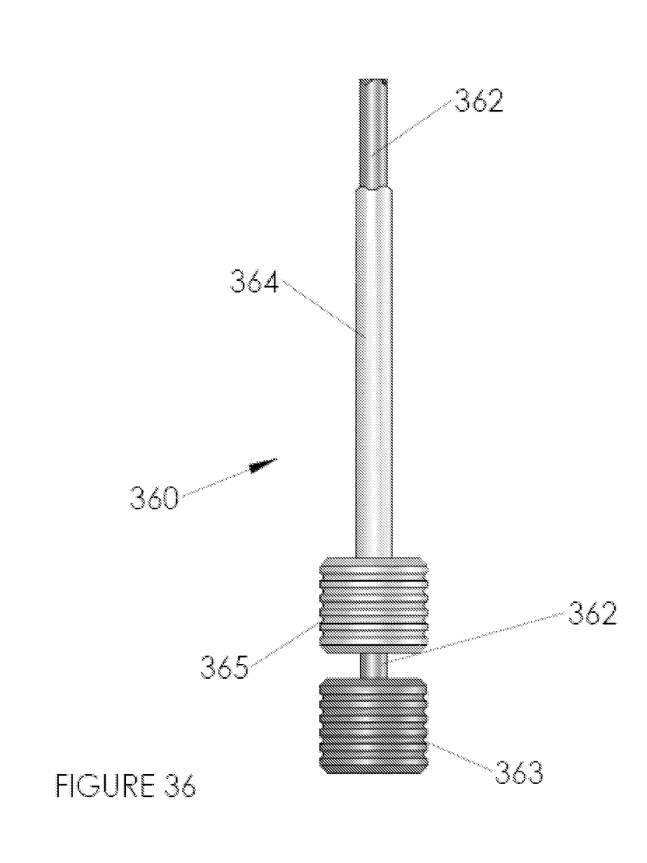

Various of the embodiments, such as illustrated by 150 in FIG. 15 and 190 in FIG. 19, require an operator or surgeon to manipulate mechanisms located in the disc nucleus from the proximal end of an outer tube such as tube 168 in FIG. 16. FIG. 36 shows one mechanism 360 to provide translational motion from a proximal location to the distal end of a tube or sheath. A first grip 363 is connected to inner tube 362 which can be slid forward and back within outer tube 364. A second grip 365 is connected to outer tube 364 to hold it immobile while inner tube 362 is moved. Second grip 365 also aids the operator to position the distal end (not shown) of the outer tube 364 within the nucleus 20. Outer tube 364 may be contiguous with or connected to other outer tubes in several embodiments of the invention such as 111 in FIG. 11B and 168 in FIG. 16. Grips 363 and 365 are shown with radial knurls in FIG. 36 as an example of an aid in handling the mechanism while an operator is wearing gloves in a wet environment. Grips 363 and 365 may be bonded chemically (e.g. by adhesive or chemical welding) or mechanically (e.g. by heat, interference fit or ultrasonic welding) to respective tubes 362 and 364. Or the grips may be formed integrally with the tubes by injection molding or a similar technique.

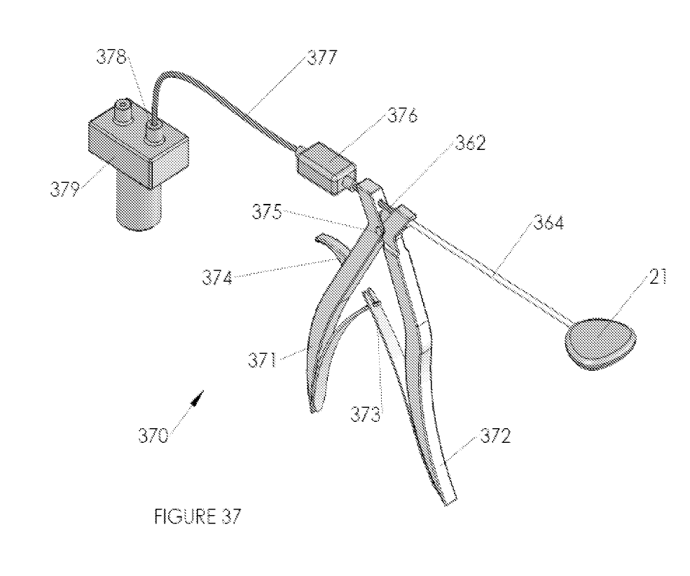

FIG. 37 illustrates another mechanism 370 to provide controlled translational motion at the proximal end of an invention as described herein. Handles 371 and 372 fit within the palm of an operators hand. Thumb guide 374 helps to ensure proper positioning while wearing gloves or in a moist environment. When the operator closes her hand the handles 371 and 372 rotate about a pivot 375 drawing inner tube 362 through outer tube 364 and partially or completely withdraws the distal end of the mechanism at the distal end of inner tube 362 from the space defined by the disc annulus 21. A spring 373 forces the handles 371 and 372 apart when the operator releases hand pressure. Inner tube 362 provides a vacuum pathway to the mechanism within the annulus space 21. A reservoir 376 to capture material evacuated through the inner tube 362 is connected at the distal end of inner tube 362 and to a flexible tube 377. The flexible tube is connected to an inlet port 378 on a vacuum pump 379.

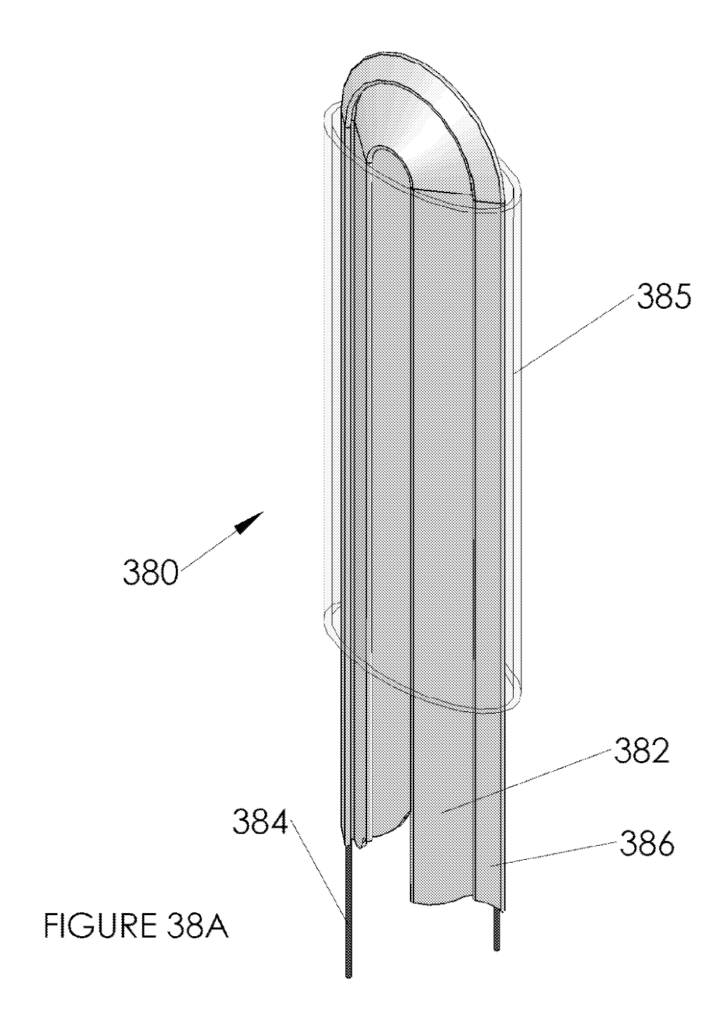

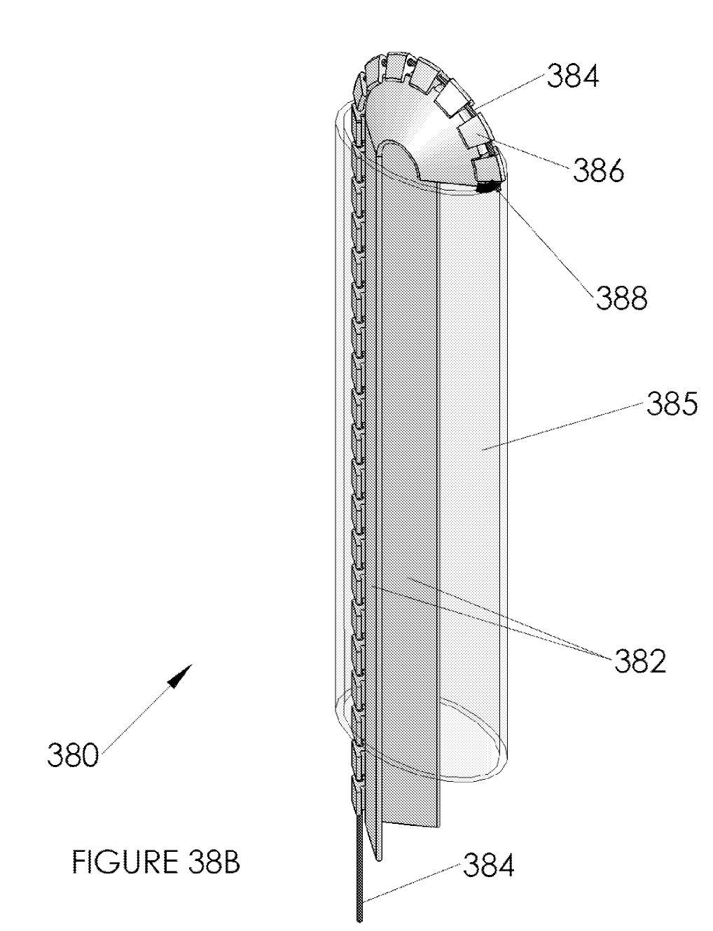

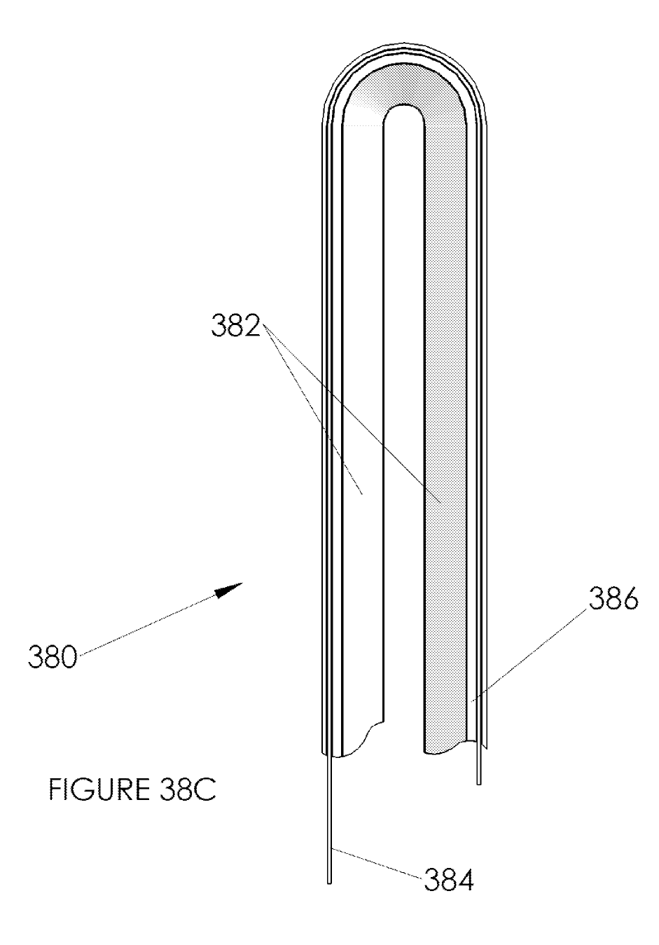

In an alternative embodiment 380 of the invention, shown in FIGS. 38A and 38C, a wiper is advanced into the nucleus space from the distal end of a hollow insertion tube. The wiper is comprised of plow blades 382 that are reinforced by shorter, support blades 386 and deployed by a wire 384. The plow blades 382 are preferably formed of a soft polymer that will not harm the annulus 21 or vertebral endplates. The support blades 386 are preferably formed with reinforcing tabs and a narrowed front edge. As illustrated in FIG. 38C, the support blade 386 may be formed with tabs spaced along the wiper rather than being continuous to facilitate easier advancement. As the wiper is advanced into the nucleus space with a wire 384 the plow blades 382 are drawn together allowing easier passage of the wiper. After the wiper is fully deployed in the nucleus space it will be in contact with essentially all of the inside edge of the annulus. The nucleus space may then be readily imaged by x-ray because the wire 384 or some other portion of the wiper is deliberately radiopaque.

Retraction of the wiper into the into an insertion tube 385 causes the blades on the wiper to spread and make continuous contact with the vertebral endplates. The support blade 386 serves to prevent the plow blade 382 from collapsing in the distal direction. Nucleus material is pulled toward the insertion tube 385 by retraction of the wiper and removed through the tube by suction.

Characteristics and advantages of the invention covered by this document have been set forth in the foregoing description. This disclosure is only illustrative in many respects. Changes can be made in details without exceeding the scope, or departing from the spirit, of the invention. The inventors' scope is defined in the language in which the claims are expressed.

We claim:

1. A method for removing nucleus pulposus from an intervertebral disc comprising:
inserting a hollow tube comprising at least one distal opening into the nucleus space of the intervertebral disc,
applying negative pressure to the hollow tube;
inflating a balloon within the nucleus space; and
manipulating inflation of the balloon and suction tube position to move the hollow tube through the nucleus space to remove nucleus material from the intervertebral disc through the hollow tube.

2. The method of claim 1 further comprising the step of providing a plurality of balloons that may be inflated separately to guide the suction tube.

3. The method of claim 1 wherein the suction tube further comprises a plurality of distal openings.

4. The method of claim 3 further comprising the step of selectively opening and closing openings of the plurality of distal openings.

5. The method of claim 4 wherein the step of opening and closing the distal openings cuts nucleus material as it enters the distal openings.

6. The method of claim 1 further comprising the step of bending the suction tube to proceed through a path conforming to the nucleus space.

7. The method of claim 6 wherein inflation of the balloon bends the suction tube.

8. The method of claim 6 wherein applying tension to a steering wire embedded in the suction tube bends the suction tube.

9. The method of claim 1 further comprising the step of providing ridges on the suction tube approximately perpendicular to the direction of motion of the suction tube.

10. The method of claim 9 further comprising the step of extending and retracting the ridges on the suction tube to maintain contact with endplates of vertebrae that contain the intervertebral disc.

11. A device for removing nucleus pulposus from an intervertebral disc comprising:
an elongate member comprising a plurality of hollow lumens;
a first opening formed in one side of the distal portion of said elongate member, said first opening in fluid communication with a first of said lumens;
an elastic membrane sealed to said side of the exterior of said elongate member and covering said first opening;
a controllable source of fluid pressure in fluid communication with said first lumen at the proximal end of said elongate member;
a second opening formed in the distal portion of said elongate member on the side substantially opposite the first opening, said opening in fluid communication with a second of said lumens; and
a controllable source of vacuum in fluid communication with said second lumen at the proximal end of said elongate member.

12. The device of claim 11 wherein said second lumen has a substantially greater cross-section area than said first lumen.

13. The device of claim 11 further comprising an opening at the distal end of said elongate member, said opening in communication with said second lumen.

14. The device of claim 11 further comprising a soft tip at the distal end of said elongate member.

15. The device of claim 11 further comprising a plurality of openings in the distal portion of said elongate member, said openings located on substantially the same side of said elongate member as said second opening, said plurality of openings in fluid communication with said second lumen.

16. The device of claim 15 further comprising:
a hollow tube rotatably located within said second lumen, said hollow tube having an outside diameter approximately the same as the inside diameter of said second lumen;
a plurality of holes formed in the distal portion of said hollow tube;
wherein said holes are equal in number to said openings and are formed at the same longitudinal position as said openings;
wherein said holes are formed at different circumferential positions of said hollow tube; and
wherein rotation of said hollow tube serves to alternately obstruct and uncover said openings in communication with said second lumen of said elongate member.

17. The device of claim 10 wherein the outside diameter of said elongate tube is preferably between 4 and 7 mm.

18. The device of claim 17 wherein the diameter of said second lumen is preferably between 2.5 and 4 mm.
